(19) United States
(12) Patent Application Publication
(10) Pub. No.: US 2006/0218670 A1
(43) Pub. Date: Sep. 28, 2006

(54) Compositions and methods for production of sialylated glyoproteins in plants

(76) Inventors: Lokesh Joshi, Tempe, AZ (US); Miti M. Shah, Scottsdale, AZ (US)

Correspondence Address: NEEDLE & ROSENBERG, P.C., SUITE 1000, 999 PEACHTREE STREET, ATLANTA, GA, 30309-3915, US

(21) Appl. No.: 11/212,506

(22) Filed: Aug. 25, 2005

Related U.S. Application Data
(63) Continuation-in-part of application No. 10/545,252, filed on Feb. 27, 2006.
(60) Provisional application No. 60/446,477, filed on Feb. 11, 2003.

(51) Int. Cl. A01H 1/00; C07H 21/04; C12N 15/82; C12N 5/04

(52) U.S. Cl. 800/284; 800/288; 435/419; 435/468; 536/23.2

(57) ABSTRACT

Disclosed are methods and compositions related to sialylation of glycoproteins.

### CROSS-REFERENCE TO RELATED APPLICATIONS

This application is a continuation-in-part of copending application Ser. No. 10/545,252, entitled “Sialylation of Glycoprotiens in Plants” by Joshi et al, filed Aug. 10, 2005, which is the National Stage of International Application No. PCT/US2004/004281, entitled “Sialylation of Glycoproteins in Plants” by Joshi et al, filed Feb. 11, 2004, which claims benefit of U.S. Provisional Application No. 60/446,477, entitled “Mammalian-Like Sialyated Glycoproteins in Plants”, filed Feb. 11, 2003, by Joshi et al, which are all herein incorporated by reference in their entirety.

### BACKGROUND

Glycosylation is one of the most frequently occurring and important post-translational modifications of proteins. Most cell surface and secreted proteins are glycosylated in the endoplasmic reticulum (ER) and Golgi by covalent attachment of sugar residues to asparagine (Asn, N-glycans) or to serine/threonine (Ser/Thr, O-glycans) residues of the proteins (A. Varki, in Essentials of Glycobiology J. E. Ajit Varki R C, Hudson Freeze, Gerald Hart, Jamey Marth, Ed. (Cold Spring Harbor Laboratory Press, New York, 1999) pp. 85-100; A. Varki, in Essentials of Glycobiology J. E. Ajit Varki, Hudson Freeze, Gerald Hart, Jamey Marth, Ed. (Cold Spring Harbor Laboratory Press, New York, 1999) pp. 85-100). In vertebrates, Sialic acid (SA) residues typically occupy the non-reducing terminal of most complex-type oligosaccharide chains on glycoproteins and glycolipids. In mammals, the non-reducing terminal SA is essential for, among other functions, intermolecular communication and extended half-life of the glycoconjugates in the circulation.

### SUMMARY

Disclosed are compositions and methods related to sialylation of glycoproteins.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the two most common SAs Neu5Ac (top) and Neu5Gc (bottom) differ in their substitution at C-5.

FIG. 2 shows the reference pathway describing SA biosynthetic and transfer to glycoconjugates.

FIG. 3 shows N- and O-linked plant glycans with potential sialylation sites.

FIG. 4A shows sialoglycoproteins from A. thaliana extract and controls were detected by SNA and MAA. FIG. 4B shows sialoglycoproteins from A (lane 1) were affinity purified through SNA and MAA columns. Alpha 2,3 sialidase predigestion resulting in no lectin binding.

FIG. 5 shows reverse-phase C18 chromatographs of DMB-SA standards (above) and A. thaliana-derived SAs (below).

FIG. 6 shows MALDI MS spectra of standard and plant cell derived DMB-Neu5Ac and DMB-Neu5Gc.

FIG. 7 shows formaldehyde-fixed and Triton X-100 permeabilized A. thaliana cells (above) and protoplasts (below) stained with TTM, SNA or MAA lectins shows the absence of SAs on cell wall but presence on and/or within the protoplast.

FIG. 8 shows protein sequence alignment and phylogram of the Sialylmotif L and Sialylmotif S of well characterized mammalian α-2-3, 2-6 and 2-8 type STs with A. thaliana and rice sequences.

FIG. 9 shows ST activity of the recombinant N. benthamiana expressed At3g48820. (A) Western blot using anti-fetuin antibodies showing the change in mobility of asialofetuin due to sialylation catalyzed by the recombinant At3g48820. Empty vector transformed plant used as control. Mobility of commercially available fetuin also shown. (B) Phosphorimager-derived image of dot blot showing transfer of radioactivity from CMP-SA to asialofetuin catalyzed by recombinant At3g48820.

FIG. 11 shows FACS analysis of the binding of linkage specific lectins SNA and MAA to cell surface sialoglyconjugates in At1g08660, At1g08280, and At1g08280 transfected CHO 2A10 cells, as compared to β-glucuronidase gene transfected control 2A10. A shift in the profile of the line compared to the outline indicates enhanced binding of lectin to the respective cells.

FIG. 12 shows western blot with anti-His antibody on soluble and microsomal protein fractions derived from S. cerevisiae transformed with constructs for expression of His-tagged At08660 and At1g08280. Expected size of recombinant proteins are about 50 kDa.

FIG. 13 shows gene expression of the A. thaliana CMP-SA transporter (At5g41760) in various tissues quantified using Real-Time RT-PCR. Transcript levels of At5g41760 have been normalized with that of Histone to account for sample to sample variations. Error bars indicate SEM (n=3).

FIG. 14 shows incorporation of ³H-GlcN and ³H-ManNAc in sialic acids as analyzed using Biogel P2 size exclusion chromatography after releasing SAs from A. thaliana glycoconjugates using mild acid hydrolysis.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular vector is disclosed and discussed and a number of vector components including the promoters are discussed, each and every combination and permutation of promoters and other vector components and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Provided herein is a method for producing recombinant sialylated glycoproteins, comprising administering a nucleic acid encoding the protein to a cell comprising a plant sialylating enzyme.

1. Sialic Acid Metabolism

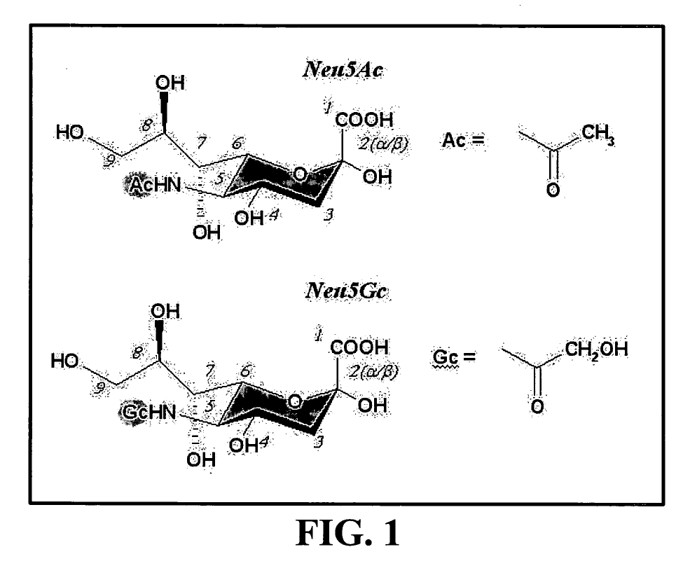

“Sialic acid” or “SA” means all of the naturally occurring structures of sialic acid including 5-acetoamido-3,5-dideoxy-D-glycero-D-galacto-nonulopyranosylonic acid (“Neu5Ac”) and the naturally occurring analogues of Neu5Ac, including N-glycolyl neuraminic acid (Neu5Gc) and 9-O-acetyl neuraminic acid (Neu5,9Ac₂), which are compatible with the selected sialyltransferase. Sialic acids (SA) are a diverse family of nine-carbon keto-sugar acids (FIG. 1) (T. Angata, A. Varki, Chemical Reviews 102, 439-469 (FEB, 2002)). More than 50 different species of SAs are found in nature, the most common being Neu5Ac (in this proposal the terms Neu5Ac and SA are used synonymously). Other forms of SA are metabolically derived from Neu5Ac by hydroxylation, O-acetylation, lactylation, methylation, sulfation or phosphorylation (T. Angata, A. Varki, Chemical Reviews 102, 439-469 (FEB, 2002)). For example, Neu5Gc, the second most common SA after Neu5Ac, is synthesized from CMP-Neu5Ac in the cytoplasm by CMP-Neu5Ac hydroxylase (L. Shaw, R. Schauer, Biological Chemistry Hoppe-Seyler 369, 477-486 (1988); M. Gollub, R. Schauer, L. Shaw, Comparative Biochemistry and Physiology Part B 120, 605-615 (1998)).

Naturally occurring sialic acids which are recognized by a particular α2,3-sialyltransferase so as to bind to the enzyme and are then available for transfer to an appropriate acceptor oligosaccharide structure are said to be compatible with the sialyltransferase.

The term “analogues of sialic acid” refers to analogues of naturally occurring structures of sialic acid including those wherein the sialic acid unit has been chemically modified so as to introduce, modify and/or remove one or more functionalities from such structures. For example, such modification can result in the removal of an OH functionality, the introduction of an amine functionality, the introduction of a halo functionality, and the like. Certain analogues of sialic acid are known in the art and include, by way of example, 9-azido-Neu5Ac, 9-amino-Neu5Ac, 9-deoxy-Neu5Ac, 9-fluoro-Neu5Ac, 9-bromo-Neu5Ac, 8-deoxy-Neu5Ac, 8-epi-Neu5Ac, 7-deoxy-Neu5Ac, 7-epi-Neu5Ac, 7,8-bis-epi-Neu5Ac, 4-O-methyl-Neu5Ac, 4-N-acetyl-Neu5Ac, 4,7-di-deoxy-Neu5Ac, 4-oxo-Neu5Ac, 3-hydroxy-Neu5Ac, 3-fluoro-Neu5Ac acid as well as the 6-thio analogues of Neu5Ac.

The carboxyl group at C-1 of SA is typically ionized at physiological pH, giving it a negative charge. Commonly, SA is linked via α-linkages between the anomeric C-2 of SA and the C-3 or C-6 of Gal residues or C-6 of GalNAc residues. The anomeric C-2 of SA can also link to C-8 of another SA, yielding polysialic acid structures such as colominic acid and neural cell adhesion molecules (N-CAMs). The combination of different substituents and linkages lends structural and functional diversity to SAs and enables multiple presentations of these residues on glycoproteins and glycolipids.

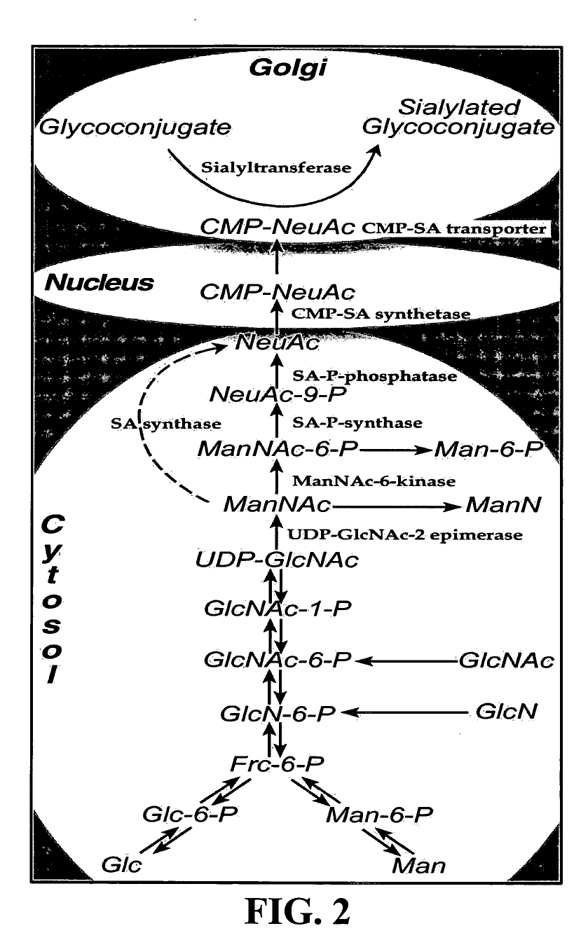

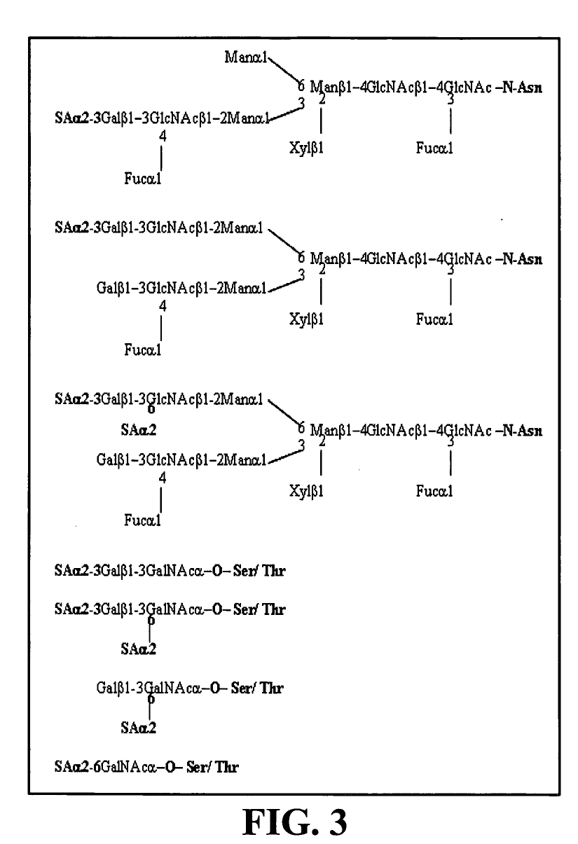

In animals, SA metabolism can be divided into three distinct processes: the synthesis of Neu5Ac in the cytoplasm and its activation to CMP-Neu5Ac in the nucleus; the transfer of Neu5Ac from CMP-Neu5Ac to the appropriate oligosaccharide acceptor (FIG. 2); and the removal and degradation of Neu5Ac, primarily in the lysosome (S. R. Reutter W, Stehling P, Baum O., in Glycosciences-Status and Perspectives. H.-J. Gabius, S. Gabius, Eds. Chapman Hall, Weinheim, 1997. pp. 245-259).

De novo synthesis of CMP-Neu5Ac is the result of a complex pathway that involves multiple steps in the cytosol beginning with glucose (FIG. 2) (O. T. Keppler et al., Science 284, 1372-1376 (1999)). The major difference between the animal and bacterial SA pathway is additional phosphorylation and dephosphorylation steps in mammalian cells (S. M. Lawrence et al., Journal of Biological Chemistry 275, 17869-17877 (2000)).

Neu5Ac made in the cytoplasm is activated by CTP to form the nucleotide sugar CMP-Neu5Ac (CMP-SA) in the nucleus, a reaction catalyzed by CMP-Neu5Ac synthetase (A. P. Corfield, R. Schauer, M. Wember, Biochemistry 177, 1-7 (1979)). Finally, CMP-Neu5Ac is transported to the Golgi apparatus and pumped across its membranes by the action of a specific antiporter, CMP-Neu5Ac (CMP-SA) transporter. Transport of nucleotide-sugars to the appropriate compartment of ER or Golgi apparatus is a prerequisite to a successful glycosylation reaction. Nucleotide-sugar transporters are Golgi membrane resident hydrophobic proteins and exist as functional dimers (R. Gerardy-Schahn, S. Oelmann, H. Bakker, Biochimie 83, 775-782 (AUG, 2001)). In animals, the CMP-SA transporter facilitates transport of CMP-SA to the Golgi lumen in a non-energy-dependent fashion. Mammalian cells lacking the CMP-SA transporter make incomplete sugar chains. This suggests that glycosylation may be partially controlled by regulating the transporter, thereby regulating the amount of nucleotide sugar available in the Golgi (S. Oelmann, P. Stanley, R. Gerardy-Schahn, Journal of Biological Chemistry 276, 26291-26300 (2001)).

Once CMP-SA is transported into the Golgi lumen, the transfer of SA onto lipid and protein bound oligosaccharides, and polysaccharides is facilitated by a large enzyme family of acceptor-specific sialyltransferases. “Sialyltransferase” or “ST” refers to those enzymes which transfer a compatible naturally occurring sialic acid, or synthetic analogs thereof, activated as its cytidine monophosphate (CMP) derivative, to the terminal oligosaccharide structures of glycolipids or glycoproteins (collectively glycoconjugates) and include enzymes produced from microorganisms genetically modified so as to incorporate and express all or part of the sialyltransferase gene obtained from another source, including mammalian sources. Numerous sialyltransferases have been identified in the literature with the different sialyltransferases generally being distinguished from each other by the terminal saccharide units on the glycoconjugates which accept the transferase. The linkage-specific transfer of SAs to glycoprotein-linked oligosaccharides is catalyzed by STs (e.g., ST3Gal, ST6Gal and ST8SA families), to α2,3, α2,5, α2,6, α2,8, and α2,9 positions. In animals, the expression of STs is species, organ, and cellular physiology dependent (N. Taniguchi, Honke K, Fukuda M., Handbook of glycosyltransferases and related genes. (Springer-Verlag, Tokyo, 2002)).

2. Plant Sialylating Enzymes

By “sialylating enzyme” is meant any enzyme involved in the synthesis, activation, transport, or transfer of sialic acid during the sialylation of a glycoconjugate. Thus, the plant sialylating enzyme of the provided method can be a CMP-sialic acid transporter. Thus, the cell of the provided method can comprise a nucleic acid encoding plant CMP-sialic acid transporter. As disclosed herein, the plant CMP-sialic acid transporter of the provided method can comprise the amino acid sequence identified in Accession No. At5g41760, BT004304, At3g59360, or AF360241. In one aspect of the provided method, the nucleic acid has sequence set forth in SEQ ID NO:1 or 2. In another aspect of the provided method, the nucleic acid hybridizes to SEQ ID NO:1 or 2 under stringent conditions. In another aspect of the provided method, the nucleic acid has at least 40%, 50%, 60%, 70%, 75%, 80%, 85%, 90%, 95% or 100% identity to the sequence set forth in SEQ ID NO:1 or 2 under stringent conditions. In another aspect of the provided method, the nucleic acid encodes a polypeptide with at least 40%, 50%, 60%, 70%, 75%, 80%, 85%, 90%, 95% or 100% identity to the sequence set forth in SEQ ID NO:6 or 7. Said encoded polypeptide can comprise conservative mutations, deletions, substitutions, or additions.

The plant sialylating enzyme of the provided method can be sialyltransferase. Thus, the cell of the provided method can comprise a nucleic acid encoding plant sialyltransferase. As disclosed herein, the plant sialyltransferase of the provided method can comprise the amino acid sequence identified in Accession No. At3g48820, AY080589, NM_{—}202675, NM_{—}114741, AY133816, At1g08660, AY064135, NM_{—}180609, NM_{—}100739, AY124807, At1g08280, BT004583, XP_{—}506687, XP_{—}463893, BAD07616, NP_{—}915574, BAB90552, BAB63715, XP_{—}473101, CAD41185, or CAE04714. In one aspect of the provided method, the nucleic acid has sequence set forth in SEQ ID NO:3, 4 or 5. In another aspect of the provided method, the nucleic acid hybridizes to SEQ ID NO: 3, 4 or 5 under stringent conditions. In another aspect of the provided method, the nucleic acid encodes a polypeptide with at least 40%, 50%, 60%, 70%, 75%, 80%, 85%, 90%, 95%, or 100% identity to the sequence set forth in SEQ ID NO:8, 9, or 10. In another aspect of the provided method, the nucleic acid has at least 40%, 50%, 60%, 70%, 75%, 80%, 85%, 90%, 95% or 100% identity to the sequence set forth in SEQ ID NO:8, 9, or 10. In another aspect of the provided method, the nucleic acid encodes a polypeptide with at least 40%, 50%, 60%, 70%, 75%, 80%, 85%, 90%, 95%, or 100% identity to the sequence set forth in SEQ ID NO:24, 25, 26, 27, 28, 29, 30, 31, or 32. Said encoded polypeptide can comprise conservative mutations, deletions, substitutions, or additions.

Sialyltransferases contain at least three conserved domains. L (long), S (short) and VS (very short). L domain is involved in binding to CMP-SA. As an example, the L sialylmotif of At1g08280 (SEQ ID NO: 10) is located within amino acids 173 to 235. It is understood that the disclosed nucleic acid encoding a plant sialyltransferase will comprise an L domain sequence with at least 40%, 50%, 60%, 70%, 75%, 80%, 85%, 90%, 95% identity to the sequence set forth in SEQ ID NO:33 or 34.

3. Overexpression

The cell of the herein provided methods can be an Arabidopsis thaliana, Nicotiana tabacum, or Medicago stativa plant cell. As disclosed herein, these cells naturally express endogenous sialylating enzymes at basal levels. Other plant cells are also considered to express endogenous sialylating enzymes herein, including higher plants, ferns and algae cells.

The cell of the provided methods can also be any cell that is overexpressing plant sialylating enzymes. Thus, the cell can be a plant cell expressing native plant sialylating enzymes at above-basal levels. For example, the cell can be an Arabidopsis thaliana, Nicotiana tabacum, or Medicago stativa plant cell that is overexpressing a plant sialylating enzyme. In certain embodiments, the cell can be any photosynthetic eukaryotic organism. Thus, the cell can be an algal cell.

As used herein, “overexpress” means that the amount of mRNA encoding an enzyme in a transformed cell is higher than the amount of mRNA encoding the enzyme expressed in a non-transformed cell. Thus, the amount of enzyme produced by a cell can be at least 50%, 60%, 70%, 80%, 90%, 100%, 150%, 200%, or 500% greater than basal levels. As an example, the nucleic acid encoding the plant sialylating enzyme can be functionally-linked to a non-native or modified expression control sequence (e.g., promoter). By “functionally-linked” is meant such that the promoter can promote expression of the nucleic acid, as is known in the art, such as appropriate orientation of the promoter relative to the nucleic acid.

4. Heterologous

The cell of the provided methods can comprise a non-native plant sialylating enzyme. Thus, the cell can be a non-plant (e.g., mammalian, bacterial, insect, fungi, archeal) cell expressing non-native plant sialylating enzymes. As an example, the cell of the provided method can be a human cell comprising a heterologous nucleic acid encoding a plant sialylating enzyme disclosed herein functionally-linked to an expression control sequence.

The term “heterologous” is used herein to refer to a nucleic acid that is derived from a different cell, tissue or organism. Furthermore, the heterologous nucleic acid preferably has all appropriate sequences for expression of the nucleic acid, as known in the art, to functionally encode, i.e., allow the nucleic acid to be expressed. The nucleic acid can include, for example, expression control sequences, such as an enhancer, and necessary information processing sites, such as ribosome binding sites, RNA splice sites, polyadenylation sites, and transcriptional terminator sequences.

a) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(1) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (fungi (e.g., yeast), insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

The viral vectors can include nucleic acid sequence encoding a marker product. This marker product is used to determine if the gene has been delivered to the cell and once delivered is being expressed. Preferred marker genes are the E. coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Non-Plant Sialylating Enzymes

As disclosed herein, heterologous expression or overexpression of plant sialyltransferase, CMP-SA transporter, or a combination thereof, is sufficient to engineer a cell to produce sialylated glycoproteins. It is not a requirement to administer other recombinant glycosylating or sialylating enzymes to the cell. For example, the necessary glycosylating or sialylating enzymes can already be present at sufficient levels in the cell, such as, for example a plant cell. Further, the recombinant expression of a protein of interest can result in feedback autoregulation of endogenous glycosylating or sialylating enzymes. However, it can be advantageous to administer other recombinant glycosylating or sialylating enzymes to the cell in combination with the plant sialyltransferase, CMP-SA transporter, or a combination thereof. Thus, the herein provided plant sialyltransferase and CMP-SA transporter can be used in any combination with other known or newly discovered glycosylating or sialylating enzymes.

Thus, the cell of the provided method can comprise exogenous sialylating enzymes. The cell can comprise a nucleic acid encoding bacterial sialic acid (SA) synthase. Thus, the cell can comprise a nucleic acid having the sequence set forth in SEQ ID NO:23. The cell can comprise a nucleic acid encoding a mammalian CMP-SA-synthetase. Thus, the cell can comprise a nucleic acid having the sequence set forth in SEQ ID NO:21 or a nucleic acid encoding the amino sequence set forth in SEQ ID NO:22. The cell can comprise a nucleic acid encoding a mammalian SA-P-phosphatase. The cell can comprise a nucleic acid encoding a mammalian SA-P-synthase. Thus, the cell can comprise a nucleic acid having the sequence set forth in SEQ ID NO:19 or a nucleic acid encoding the amino sequence set forth in SEQ ID NO:20. The cell can comprise a nucleic acid encoding a mammalian UDP-GlcNAc-2-epimerase/ManNAc kinase. Thus, the cell can comprise a nucleic acid having the sequence set forth in SEQ ID NO:17 or a nucleic acid encoding the amino sequence set forth in SEQ ID NO:18. The cell can comprise a nucleic acid encoding a mammalian GlcNAc-2-epimerase. Thus, the cell can comprise a nucleic acid having the sequence set forth in SEQ ID NO:15 or a nucleic acid encoding the amino sequence set forth in SEQ ID NO:16.

It is understood that the cell comprises CMP-NeuAc (CMP-SA), or an analog thereof. Thus, the cell can comprise the enzymes necessary to synthesize CMP-SA from UDP-GlcNAc (e.g., mammalian CMP-SA-synthetase, SA-P-phosphatase, SA-P-synthase, ManNAc-6-kinase, and UDP-GlcNAc-2-epimerase). Alternatively, CMP-SA, or an analog thereof, can be administered exogenously to the cell. Likewise, UDP-GlcNAc can be synthesized by the cell or provided exogenously.

6. Recombinant Proteins

Provided for use in the present method is nucleic acid encoding a protein in which oligosaccharide processing is desired. Below is a non-limiting list of selected cloned proteins that could be produced by the present method.

TABLE 1
Cloned structural genes.

| Gene | Clone Type* | Reference |
|---|---|---|
| activin | porcine-cDNA | Mason AJ, Nat, 318: 659, 1985 |
| adenosine deaminase | h-cDNA | Wiginton DA, PNAS, 80: 7481, 1983 |
| angiotensinogen I | r-cDNA | Ohkubo H, PNAS, 80: 2196, 1983 |
| | r-gDNA | Tanaka T, JBC, 259: 8063, 1984 |
| antithrombin III | h-cDNA | Bock SC, NAR 10: 8113, 1982 |
| | h-cDNA and gDNA | Prochownik EV, JBC, 258: 8389, 1983 |
| antitrypsin, alpha I | h-cDNA | Kurachi K, PNAS, 78: 6826, 1981 |
| | h-gDNA | Leicht M, Nat, 297: 655, 1982 |
| | RFLP | Cox DW, AJHG, 36: 134S, 1984 |
| apolipoprotein A-I | h-cDNA, h-gDNA | Shoulders CC, NAR, 10: 4873, 1982 |
| | RFLP | Karathanasis SK, Nat, 301: 718, 1983 |
| | h-gDNA | Kranthanasis SK, PNAS, 80: 6147, 1983 |
| apolipoprotein A-II | h-cDNA | Sharpe CR, NAR, 12: 3917, 1984 |
| | Chr | Sakaguchi AY, AJHG, 36: 207S, 1984 |
| | h-cDNA | Knott TJ, BBRC, 120: 734, 1984 |
| apolipoprotein C-I | h-cDNA | Knott TJ, NAR, 12: 3909, 1984 |
| apolipoprotein C-II | h-cDNA | Jackson CL, PNAS, 81: 2945, 1984 |
| | h-cDNA | Mykelbost O, JBC, 259: 4401, 1984 |
| | h-cDNA | Fojo SS, PNAS, 81: 6354, 1984 |
| | RFLP | Humphries SE, C Gen, 26: 389, 1984 |
| apolipoprotein C-III | h-cDNA and gDNA | Karanthanasis SK, Nat, 304: 371, 1983 |
| | h-cDNA | Sharpe CR, NAR, 12: 3917, 1984 |
| apolipoprotein E | h-cDNA | Breslow JL, JBC, 257: 14639, 1982 |
| atrial natriuretic factor | h-cDNA | Oikawa S, Nat, 309: 724, 1984 |
| | h-cDNA | Nakayama K, Nat, 310: 699, 1984 |
| | h-cDNA | Zivin RA, PNAS, 81: 6325, 1984 |
| | h-gDNA | Seidman CE, Sci, 226: 1206, 1984 |
| | h-gDNA | Nemer M, Nat, 312: 654, 1984 |
| | h-gDNA | Greenberg BI, Nat, 312: 665, 1984 |
| chorionic | h-cDNA | Fiddes JC, Nat, 281: 351, 1981 |
| gonadotropin, alpha chain | RFLP | Boethby M, JBC, 256: 5121, 1981 |
| chorionic | h-cDNA | Fiddes JC, Nat, 286: 684, 1980 |
| gonadotropin, | h-gDNA | Boorstein WR, Nat, 300: 419, 1982 |
| beta chain | h-gDNA | Talmadge K, Nat, 307: 37, 1984 |
| chymosin, pro (rennin) | bovine-cDNA | Harris TJR, NAR, 10: 2177, 1982 |
| complement, factor B | h-cDNA | Woods DE, PNAS, 79: 5661, 1982 |
| | h-cDNA and gDNA | Duncan R, PNAS, 80: 4464, 1983 |
| complement C2 | h-cDNA | Bentley DR, PNAS, 81: 1212, 1984 |
| | h-gDNA (C2, C4, and | Carroll MC, Nat, 307: 237, 1984 |
| | B) | |
| complement C3 | m-cDNA | Domdey H, PNAS, 79: 7619, 1983 |
| | h-gDNA | Whitehead AS, PNAS, 79: 5021, 1982 |
| complement C4 | h-cDNA and gDNA | Carroll MC, PNAS, 80: 264, 1983 |
| | h-cDNA | Whitehead AS, PNAS, 80: 5387, 1983 |
| complement C9 | h-cDNA | DiScipio RC, PNAS, 81: 7298, 1984 |
| corticotropin | Sheep-cDNA | Furutani Y, Nat, 301: 537, 1983 |
| releasing factor | h-gDNA | Shibahara S, EMBO J, 2: 775, 1983 |
| epidermal growth factor | m-cDNA | Gray A, Nat, 303: 722, 1983 |
| | m-cDNA | Scott J, Sci, 221: 236, 1983 |
| | h-gDNA | Brissenden JE, Nat, 310: 781, 1984 |
| epidermal growth factor | h-cDNA and Chr | Lan CR, Sci, 224: 843, 1984 |
| receptor, oncogene c-erb B | | |
| epoxide dehydratase | r-cDNA | Gonzalez FJ, JBC, 256: 4697, 1981 |
| erythropoietin | h-cDNA | Lee-Huang S, PNAS, 81: 2708, 1984 |
| esterase inhibitor, | h-cDNA, | Stanley KK, EMBO J, 3: 1429, 1984 |
| C1 factor VIII | h-cDNA and gDNA | Gitschier J, Nat, 312: 326, 1984 |
| | h-cDNA | Toole JJ, Nat, 312: 342, 1984 |
| factor IX, Christmas | h-cDNA | Kutachi K, PNAS, 79: 6461, 1982 |
| factor | | |
| | h-cDNA | Choo KH, Nat, 299: 178, 1982 |
| | RFLP | Camerino G, PNAS, 81: 498, 1984 |
| | h-gDNA | Anson DS, EMBO J, 3: 1053, 1984 |
| factor X | h-cDNA | Leytus SP, PNAS, 81: 3699, 1984 |
| fibrinogen A alpha, | h-cDNA | Kant JA, PNAS, 80: 3953, 1983 |
| B beta, gamma | h-gDNA (gamma) | Fornace AJ, Sci, 224: 161, 1984 |
| | h-cDNA (alpha gamma) | Imam AMA, NAR, 11: 7427, 1983 |
| | h-gDNA (gamma) | Fornace AJ, JBC, 259: 12826, 1984 |
| gatrin releasing peptide | h-cDNA | Spindel ER, FNAS, 81: 5699, 1984 |
| glucagon, prepro | hamster-cDNA | Bell GI, Nat, 302: 716, 1983 |
| | h-gDNA | Bell GI, Nat, 304: 368, 1983 |
| growth hormone | h-cDNA | Martial JA, Sci, 205: 602, 1979 |
| | h-gDNA | DeNoto FM, NAR, 9: 3719, 1981 |
| | GH-like gene | Owerbach D, Sci, 209: 289, 1980 |
| growth hormone RF, | h-cDNA | Gubler V, PNAS, 80: 4311, 1983 |
| somatocrinin | h-cDNA | Mayo KE, Nat, 306: 86: 1983 |
| hemopexin | h-cDNA | Stanley KK, EMBO J, 3: 1429, 1984 |
| inhibin | porcine-cDNA | Mason AJ, Nat, 318: 659, 1985 |
| insulin, prepro | h-gDNA | Ullrich a, Sci, 209: 612, 1980 |
| insulin-like growth factor I | h-cDNA | Jansen M, Nat, 306: 609, 1983 |
| | h-cDNA | Bell GI, Nat, 310: 775, 1984 |
| | Chr | Brissenden JE, Nat, 310: 781, 1984 |
| insulin-like growth factor | h-cDNA | Bell GI, Nat, 310: 775, 1984 |
| II | | |
| | h-gDNA | Dull TJ, Nat, 310: 777, 1984 |
| | Chr | Brissenden JE, Nat, 310: 781, 1984 |
| interferon, alpha | h-cDNA | Maeda S, PNAS, 77: 7010, 1980 |
| (leukocyte), multiple | | |
| | h-cDNA (8 distinct) | Goeddel DV, Nat, 290: 20, 1981 |
| | h-gDNA | Lawn RM, PNAS, 78: 5435, 1981 |
| | h-gDNA | Todokoro K, EMBO J, 3: 1809, 1984 |
| | h-gDNA | Torczynski RM, PNAS, 81: 6451, 1984 |
| interferon, beta | h-cDNA | Taniguchi T, Gene, 10: 11, 1980 |
| (fibroblast) | | |
| | h-gDNA | Lawn RM, NAR, 9: 1045, 1981 |
| | h-gDNA (related) | Sehgal PB, PNAS, 80: 3632, 1983 |
| | h-gDNA (related) | Sagar AD, Sci, 223: 1312, 1984 |
| interferon, gamma | h-cDNA | Gray PW, Nat, 295: 503, 1982 |
| (immune) | h-gDNA | Gray PW, Nat, 298: 859, 1982 |
| interleukin-1 | m-cDNA | Lomedico PT, Nat, 312: 458, 1984 |
| interleukin-2, T-cell | h-cDNA | Devos R, NAR, 11: 4307, 1983 |
| growth factor | h-cDNA | Taniguchi T, Nat, 302: 305, 1983 |
| | h-gDNA | Hollbrook NJ, PNAS, 81: 1634, 1984 |
| | Chr | Siegel LF, Sci, 223: 175, 1984 |
| interluekin-3 | m-cDNA | Fung MC, Nat, 307: 233, 1984 |
| kininogen, two forms | bovine-cDNA | Nawa H, PNAS, 80: 90, 1983 |
| | bovine-cDNA and | Kitamura N, Nat, 305: 545, 1983 |
| | gDNA | |
| luteinizing hormone, beta | h-gDNA and Chr | Talmadge K, Nat, 207: 37, 1984 |
| subunit | | |
| luteinizing hormone | h-cDNA and gDNA | Seeburg PH, Nat, 311: 666, 1984 |
| releasing hormone | | |
| lymphotoxin | h-cDNA and gDNA | Gray PW, Nat, 312: 721, 1984 |
| mast cell growth factor | m-cDNA | Yokoya T, PNAS, 81: 1070, 1984 |
| nerve growth factor, | m-cDNA | Scott J, Nat, 302: 538, 1983 |
| beta subunit | h-gDNA | Ullrich A, Nat, 303: 821, 1983 |
| | Chr | Franke C, Sci, 222: 1248, 1983 |
| oncogene, c-sis, | h-gDNA | Dalla-Favera R, Nat, 295: 31, 1981 |
| PGDF chain A | h-cDNA | Clarke MF, Nat, 208: 464, 1984 |
| pancreatic polypeptide | h-cDNA and | Boel B, EMBO J, 3: 909, 1984 |
| | icosapeptide | |
| parathyroid | h-cDNA | Hendy GN, PNAS, 78: 7365, 1981 |
| hormone, prepro | h-gDNA | Vasicek TJ, PNAS, 80: 2127, 1983 |
| plasminogen | h-cDNA and gDNA | Malinowski DP, Fed P, 42: 1761, 1983 |
| plasminogen | h-cDNA | Edlund T, PNAS, 80: 349, 1983 |
| activator | h-cDNA | Pennica D, Nat, 301: 214, 1983 |
| | h-gDNA | Ny T, PNAS, 81: 5355, 1984 |
| prolactin | h-cDNA | Cook NE, JBC, 256: 4007, 1981 |
| | r-gDNA | Cooke NE, Nat, 297: 603, 1982 |
| proopiomelanocortin | h-cDNA | DeBold CR, Sci, 220: 721, 1983 |
| | h-gDNA | Cochet M, Nat, 297: 335, 1982 |
| protein C | h-cDNA | Foster D, PNAS, 81: 4766, 1984 |
| prothrombin | bovine-cDNA | MacGillivray RTA, PNAS, 77: 5153, |
| | | 1980 |
| relaxin | h-gDNA | Hudson P, Nat, 301: 628, 1983 |
| | h-cDNA (2 genes) | Hudson P, EMBO J, 3: 2333, 1984 |
| | Chr | Crawford RJ, EMBO J, 3: 2341, 1984 |
| renin, prepro | h-cDNA | Imai T, PNAS, 80: 7405, 1983 |
| | h-gDNA | Hobart PM, PNAS 81: 5026, 1984 |
| | h-gDNA | Miyazaki H, PNAS, 81: 5999, 1984 |
| | Chr | Chirgwin JM, SCMG, 10: 415, 1984 |
| somatostatin | h-cDNA | Shen IP, PNAS, 79: 4575, 1982 |
| | h-gDNA and Ri-IP | Naylot SI, PNAS, 80: 2686, 1983 |
| tachykinin, prepro, | bovine-cDNA | Nawa H, Nat, 306: 32, 1983 |
| substances P & K | bovine-gDNA | Nawa H, Nat, 312: 729, 1984 |
| urokinase | h-cDNA | Verde P, PNAS, 81: 4727, 1984 |
| vasoactive intestinal | h-cDNA | Itoh N, Nat, 304: 547, 1983 |
| peptide, prepro | | |
| vasopressin | r-cDNA | Schmale H, EMBO J, 2: 763, 1983 |

Key 4: *cDNA—complementary DNA; Chr—chromosome; gDNA—genomic DNA; RFLP—restriction fragment polymorphism; h—human; m—mouse; r—rat

Thus, the herein provided method can be used to produce recombinant sialylated glycoproteins. Non-limiting examples of glycoproteins that can be produced by the provided method include: activin, adenosine deaminase, angiotensinogen I, antithrombin III, antitrypsin, alpha I, apolipoprotein A-I, apolipoprotein A-II, apolipoprotein C-I, apolipoprotein C-II, polipoprotein C-III, apolipoprotein E, atrial natriuretic factor, chorionic gonadotropin, alpha chain, chorionic gonadotropin, beta chain, chymosin, pro, complement, factor B, complement C2, complement C3, complement C4, complement C9, corticotrophin releasing factor, epidermal growth factor, epidermal growth factor receptor, epoxide dehydratase, erythropoietin esterase inhibitor, C1 factor VIII, factor IX, factor X, fibrinogen, gatrin releasing peptide, glucagons, growth hormone, growth hormone RF, somatocrinin, hemopexin, inhibin, insulin, prepro, insulin-like growth factor I, insulin-like growth factor II, interferon alpha, interferon beta, interferon gamma, interleukin-1, interleukin-2, interluekin-3, kininogen, luteinizing hormone beta subunit, luteinizing hormone releasing hormone, lymphotoxin, mast cell growth factor, nerve growth factor beta subunit, oncogene c-sis, PGDF chain A, pancreatic polypeptide, parathyroid hormone, plasminogen, plasminogen activator, prolactin, proopiomelanocortin, protein C, prothrombin, relaxin, rennin, somatostatin, tachykinin, substances P & K, urokinase, vasoactive intestinal peptide, vasopressin, immunoglobuluins (e.g. recombinant antibodies), vaccine epitopess, hormones, neurotrophic factors, such as neural cell adhesion molecule (NCAM).

7. Expressing and Isolating Recombinant Proteins in Plants

The biochemical, technical and economic limitations on existing prokaryotic and eukaryotic expression systems have created substantial interest in developing new expression systems for the production of recombinant proteins. Like microbes, plant cells are inexpensive to grow and maintain, but because they are higher eukaryotes they can carry out many of the post-translational modifications that occur in human cells. Plant cells are also intrinsically safe, because they neither harbor human pathogens nor produce endotoxins. Thus, plants represent the most likely alternative to existing expression systems. With the availability and on going development of plant transformation techniques, most commercially important plant species can now be genetically modified to express a variety of recombinant proteins.

Such transformation techniques include, for example, the Agrobacterium vector system, which involves infection of the plant tissue with a bacterium (Agrobacterium) into which the foreign gene has been inserted. A number of methods for transforming plant cells with Agrobacterium are well known (Klee et al., Annu. Rev. Plant Physiol. (1987) 38:467-486; Schell and Vasil Academic Publishers, San Diego, Calif. (1989) p. 2-25; and Gatenby (1989) in Plant Biotechnology, eds. Kung, S. and Arntzen, C. J., Butterworth Publishers, Boston, Mass. p. 93-112), all of which are hereby incorporated herein by reference for their teaching of plant transforming methods.

The biolistic or particle gun method, which permits genetic material to be delivered directly into intact cells or tissues by bombarding regeneratable tissues, such as meristems or embryogenic callus, with DNA-coated microparticles has contributed to plant transformation simplicity and efficiency. The microparticles penetrate the plant cells and act as inert carriers of a genetic material to be introduced therein. Microprojectile bombardment of embryogenic suspension cultures has proven successful for the production of transgenic plants of a variety of species. Various parameters that influence DNA delivery by particle bombardment have been defined (Klein et al., Bio/Technology (1998) 6:559-563; McCabe et al., Bio/Technology (1998) 6:923-926; and Sanford, Physiol. Plant. (1990) 79:206-209), all of which are hereby incorporated herein by reference for their teaching of biolistic and particle gun methods.

Micropipette systems are also used for the delivery of foreign DNA into plants via microinjection (Neuhaus et al., Theor. Appl. Genet. (1987) 75:30-36; and Neuhaus and Spangenberg, Physiol. Plant. (1990) 79:213-217), all of which are hereby incorporated herein by reference for their teaching of micropipette systems.

Other techniques developed to introduce foreign genes into plants include direct DNA uptake by plant tissue, or plant cell protoplasts (Schell and Vasil (1987) Academic Publishers, San Diego, Calif. p. 52-68; and Toriyama et al., Bio/Technology (1988) 6:1072-1074) or by germinating pollen (Chapman, Mantell and Daniels (1985) W. Longman, London, p. 197-209; and Ohta, Proc. Natl. Acad. Sci. USA (1986) 83:715-719), all of which are hereby incorporated herein by reference for their teaching of plant transforming methods.

DNA uptake induced by brief electric shock of plant cells has also been described (Zhang et al., Plant. Cell. Rep. (1988) 7:379-384 and Fromm et al., Nature (1986) 319:791-793), all of which are hereby incorporated herein by reference for their teaching of plant transforming methods.

In addition, virus mediated plant transformation has also been extensively described. Transformation of plants using plant viruses is described, for example, in U.S. Pat. No. 4,855,237 (BGV), EP-A 67,553 (TMV), Japanese Published Application No. 63-14693, EPA 194,809, EPA 278,667, and Gluzman et al., (1988) Communications in Molecular Biology: Viral Vectors, Cold Spring Harbor Laboratory, New York, pp. 172-189. Pseudovirus particles for use in expressing foreign DNA in many hosts, including plants, have also been described, for reference, see, for example WO 87/06261. All preceding references are hereby incorporated herein by reference for their teaching of plant transforming methods.

The production of recombinant proteins and peptides in plants has been investigated using a variety of approaches including transcriptional fusions using a strong constitutive plant promoter (e.g., from cauliflower mosaic virus, Sijmons et al., Bio/Technology (1990) 8:217-221); transcriptional fusions with organ specific promoter sequences (Radke et al., Theoret. Appl. Genet. (1988) 75:685-694); and translational fusions which require subsequent cleavage of a recombinant protein (Vanderkerckove et al., Bio/Technology (1989) 7:929-932). All preceding references are hereby incorporated herein by reference for their teaching of transcriptional fusions for production of recombinant proteins in plants.

The application of such genetic transformation techniques has allowed the incorporation of a variety of important genetic traits for crop improvement and also for the biotechnological production of extractable, valuable, foreign proteins including enzymes, vaccine proteins and antibodies.

Foreign proteins that have been successfully expressed in plant cells include proteins from bacteria (Fraley et al. Proc. Natl. Acad. Sci. U.S.A (1993) 80:4803-4807), animals (Misra and Gedamu, Theor. Appl. Genet. (1989) 78:161-168), fungi and other plant species (Fraley et al. Proc. Natl. Acad. Sci. U.S.A. (1983) 80:4803-4807). Some proteins, predominantly markers of DNA integration, have been expressed in specific cells and tissues including seeds (Sen Gupta-Gopalan et al. Proc. Natl. Acad. Sci. U.S.A. (1985) 82:3320-3324; Radke et al. Theor. Appl. Genet. (1988) 75:685-694).

The nucleic acid encoding the protein of interest can be introduced into a host cell in a form where the nucleic acid is stably incorporated into the genome of the host cell. One may also introduce the nucleic acid as part of a recombinant DNA sequence capable of replication and or expression in the host cell without the need to become integrated into the host chromosome.

The nucleic acid introduced into the plant cell may also comprise sequences for regulation of transcription which are recognized by the plant cell. The regulatory sequences can comprise one or more promoter(s) of plant or viral origin or obtained from Agrobacterium tumefaciens. Thus, the nucleic acid can comprise a constitutive promoter, for example the CaMV 35S, the double 35S, the Nos or OCS promoters, or promoters specific for certain tissues such as the grain or specific for certain phases of development of the plant. The nucleic acid can comprise promoters specific for seeds, such as the promoter of the gene for napin and for the acyl carrier protein (ACP) (EP-A-0,255,378), as well as the promoters of the AT2S genes of Arabidopsis thaliana, that is to say the PAT2S 1, PAT2S2, PAT2S3 and PAT2S4 promoters (Krebbers et al., Plant Physiol., 1988, vol. 87, pages 859-866). The nucleic acid can comprise the cruciferin or phaseolin promoter or pGEA1 and pGEA6 of Arabidopsis, promoters of genes of the “em, Early Methionine labeled protein” type, which is strongly expressed during the phases of drying of the seed.

The introduction of a nucleic acid molecule(s) into the plant cell can be carried out in a stable manner either by transformation of the nuclear genome, or by transformation of the chloroplast genome of the plant cell, or by transformation of the mitochondrial genome.

For the transformation of the nuclear genome, conventional techniques may be used. All known means for introducing foreign DNA into plant cells may be used, for example Agrobacterium (e.g., Agrobacterium tumefaciens and Agrobacterium rhizogenes), electroporation, protoplast fusion, particle gun bombardment, or penetration of DNA into cells such as pollen, microspore, seed and immature embryo. Viral vectors such as the Gemini viruses or the satellite viruses may also be used as introducing means.

The introduction of the nucleic acid into the plant cell can also be carried out by the transformation of the mitochondrial or chloroplast genomes (see for example Carrer et al., Mol. Gen. Genet., 1993, 241, 49-56). Techniques for direct transformation of the chloroplasts or the mitochondria are known per se and may comprise introducing transformant DNA by the biolistic technique (Svab et al., P.N.A.S., 1990, 87, 8526-8530); integrating the transformant DNA by two homologous recombination events; and selectively removing copies of the wild-type genome during repeated cell divisions on selective medium.

a) Transgenic Plants

Chimeric or transgenic plants can be generated from transformed explants, using techniques known per se.

b) In Vitro

Unlike field-grown plants, the performance of cultured plant cells is independent of the climate, soil quality, season, day length and weather. There is no risk of contamination with mycotoxins, herbicides or pesticides and there are fewer by-products (e.g., fibers, oils, waxes, phenolics and adventitious agents). Perhaps the most important advantage of plant cells over whole plants is the much simpler procedure for product isolation and purification especially when the product is secreted into the culture medium.

Several approaches can be used for the in vitro cultivation of plant cells, including the derivation of hairy roots, shooty teratomas, immobilized cells and suspension cell cultures. Suspension cells have the advantage that they can be cultivated relatively easily in large-scale bioreactors. Suspension cell cultures have been prepared from several different plant species, including Arabidopsis thaliana, Taxus cuspidata, Catharanthus roseus and important domestic crops such as tobacco, alfalfa, rice, tomato and soybean.

Plant suspension cells are prepared by the agitation of friable callus tissue in shaker flasks or fermenters to form single cells and small aggregates. Callus is undifferentiated tissue obtained by cultivating explants on solid medium containing the appropriate mixture of plant hormones to maintain the undifferentiated state. The cells are grown in liquid culture medium containing the same hormones to promote rapid growth and prevent differentiation.

If transgenic plants expressing the recombinant protein of interest are used as the source of callus tissue, further genetic manipulation is unnecessary (that is, the callus and/or suspension does not have to be selected for transformed cells). Alternatively, wild-type cell suspensions can be transformed with recombinant plasmids either by cocultivation with Agrobacterium tumefaciens or particle bombardment.

The principles applied to the culture of microbial cells apply also to plant cells, although cell densities and growth rates are lower. Oxygen uptake rates (and thus the oxygen transfer rates the bioreactor has to deliver) are also relatively low in plant cells. For example, Taticek et al. reported an oxygen uptake rate (OUT) of 1-3.5 mmol l⁻¹ h⁻¹ in plant cell cultures, compared with ˜5-90 mmol l⁻¹ h⁻¹ in bacterial cultures. Despite these differences, conventional fermenter equipment can be modified easily to work with plant cells, and many of the fermentation strategies applied to microbial cultures can also be applied to plants.

The cells of provided methods can also be immobilized, which makes it possible to obtain a constant and prolonged production of recombinant protein. The separation of the recombinant protein and the plant biomass is also facilitated. As immobilization method, there may be mentioned immobilization in alginate or agar beads, inside polyurethane foam, or alternatively inside hollow fibers.

The cells of the provided methods can also be root cultures. The roots cultivated in vitro, in a liquid medium, are called “Hairy roots”, they are roots transformed by the bacterium Agrobacterium rhizogenes.

Thus, there are varied methods for the recombinant expression of foreign genes in plants. It is understood that one of skill in the art would be able to use the herein provided compositions and methods to produce sialylated glycoproteins in any plant. The challenges that are associated with using different plant species, such as the transformation of a plant with a gene, or purification of the protein encoded by the gene from the plant, can be overcome using standard methods known in the art and provided herein.

8. Protein Purification

The provided method can also include isolation or purification of the protein or polypeptide of interest. The term “purified recombinant heterologous protein” as used herein, is intended to refer to a recombinant heterologous protein composition, isolatable from host cells, wherein the recombinant heterologous protein is purified to any degree relative to its naturally-obtainable state, i.e., in this case, relative to its purity within a natural extract. A purified recombinant heterologous protein therefore also refers to a recombinant heterologous protein free from the environment in which it may naturally occur.

Generally, “purified” will refer to a recombinant heterologous protein composition which has been subjected to fractionation to remove various cell components. Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Methods exhibiting a lower degree of relative purification may have advantages in total recovery of protein product, or in maintaining the activity of an expressed protein. Inactive products also have utility in certain embodiments, such as, e.g., in antibody generation.

Partially purified recombinant heterologous protein fractions for use in such embodiments may be obtained by subjecting a cell extract to one or a combination of the steps described above. Substituting certain steps with improved equivalents is also contemplated to be useful. For example, it is appreciated that a cation-exchange column chromatography performed utilizing an HPLC apparatus will generally result in a greater-fold purification than the same technique utilizing a low pressure chromatography system.

Due to the advantageous economics of field-grown crops, the ability to synthesize proteins in storage organs like tubers, seeds, fruits and leaves and the ability of plants to perform many of the post-translational modifications, as previously described and as described herein, several plant expression systems are currently investigated for potential as highly effective and economically feasible systems for the production of recombinant proteins. Alternative expression approaches have been undertaken in an effort to simplify the purification procedure of the recombinant protein from the plant cells. One such system focuses on the use of seed-storage protein promoters as a means of deriving seed-specific expression. Using such a system, Vanderkerckove et al., (Bio/Technol. (1989) 7:929-932) expressed the peptide Leu-enkephalin in seeds of Arabidopsis thaliana and Brassica napus. Another system utilizing seeds as an expression host is disclosed in U.S. Pat. No. 5,888,789, which is herein incorporated by reference for the teaching of the expression and purification of recombinant proteins from plants. This system provides for the secretion of heterologous protein by malting of monocot plant seeds. The heterologous genes are expressed during germination of the seeds and isolated from a malt.

U.S. Pat. No. 5,580,768, which is herein incorporated by reference for the teaching of the expression and purification of recombinant proteins from plants, describes a method of producing a genetically transformed fluid-producing plant. The genetically transformed plant which can be for example, a rubber secreting (Hevea) plant is capable of expressing the target product in the fluid that it produces which in this case is latex. U.S. Pat. No. 5,650,554 describes the use of a class of genes called oil body protein genes, that have unique features, allowing the production of recombinant proteins that can be easily separated from other host cell components. Many additional expression systems have been described utilizing specific targeting or directing of recombinant proteins to specific plant tissues.

U.S. Pat. No. 5,474,925, which is herein incorporated by reference for the teaching of the expression and purification of recombinant proteins from plants, describes an expression construct utilizing a signal peptide translationally fused to a recombinant enzyme which targets the enzyme to the cellulose matrix of the cell wall. This enables the isolation of the enzyme along with the easily recoverable cellulose matrix. This system is utilized for the localized expression of commercially important enzymes in cotton fibers. According to this system, the expressed enzymes are recovered along with the cellulosic matter of the fibers. The enzyme-cellulose matrix recovered, is directly utilized for commercial enzymatic processes.

U.S. Pat. No. 6,331,416, which is herein incorporated by reference for the teaching of the expression and purification of recombinant proteins from plants, describes a process of expressing a recombinant protein in a plant and of isolating the recombinant protein from the plant by fusing a cellulose binding peptide to the recombinant protein, which will complex with the cellulosic matter during homogenization.

9. Method of Engineering

Also provided herein is a method for engineering plants to produce recombinant sialylated glycoproteins, comprising administering to the cell a nucleic acid encoding a plant sialylating enzyme. The plant sialylating enzyme can be CMP-sialic acid transporter. Thus, the nucleic acid can have the sequence set forth in SEQ ID NO:1 or 2. The plant sialylating enzyme can be sialyltransferase. Thus, the nucleic acid can have the sequence set forth in SEQ ID NO:3, 4 or 5.

The disclosed nucleic acid encoding a plant sialylating enzyme can be administered in combination with a nucleic acid encoding a sialylating or glycosylating enzyme, such as those provided herein. Thus, the provided method can further comprise administering to the plant cell a nucleic acid encoding an enzyme selected from the group consisting of CMP-SA-synthetase, SA-P-phosphatase, SA-P-synthase, ManNAc-6-kinase, and UDP-GlcNAc-2-epimerase.

The nucleic acid can be introduced into a cell in a form where the nucleic acid is stably incorporated into the genome of the host cell. One may also introduce the nucleic acid as part of a recombinant DNA sequence capable of replication and or expression in the host cell without the need to become integrated into the host chromosome.

10. Ex Vivo Sialylation

Also provided herein are methods of glycosylating substrates by contacting a plant sialyltransferase with a suitable sialic acid substrate acceptor and a sialic acid donor. Thus, provided is a method comprising contacting a plant sialyltransferase, or a biologically active fragment thereof, with a sialic acid acceptor substrate and a sialic acid donor under conditions effective for transfer of sialica acid from a donor molecule to a the acceptor substrate. The plant sialyltransferase of the present method can be any plant sialyltransferase disclosed herein. Useful sialic acid donors include CMP-Neu5Ac (CMP-SA). Suitable sialic acid acceptor substrates include carbohydrate groups of glycoproteins and glycolipids. Such method can be used to design and engineer molecules to contain specific sugar residues. Non-limiting examples of suitable sialic acids can be found in Table 2.

TABLE 2
Occurrence of Sialic Acids

| Compound | Abbreviation | Occurrence |
|---|---|---|
| neuraminic acid | Neu | V |
| neuraminic acid 1,5-lactam | Ne1,5lactam | V |
| 5-N-acetylneuraminic acid | Neu5Ac | V, E, Ps, Pz, |
| | | F, B |
| 5-N-acetyl-4-O-acetylneuraminic acid | Neu4,5Ac2 | V |
| 5-N-acetyl-7-O-acetylneuraminic acid | Neu5,7Ac2 | V, Pz, B |
| 5-N-acetyl-8-O-acetylneuraminic acid | Neu5,8Ac2 | V, B |
| 5-N-acetyl-9-O-acetylneuraminic acid | Neu5,9Ac2 | V, E, Pz, F, B |
| 5-N-acetyl-4,9-di-O-acetylneuraminic acid | Neu4,5,9Ac3 | V |
| 5-N-acetyl-7,9-di-O-acetylneuraminic acid | Neu5,7,9Ac3 | V, B |
| 5-N-acetyl-8,9-di-O-acetylneuraminic acid | Neu5,8,9Ac3 | V |
| 5-N-acetyl-4,7,9-tri-O-acetylneuraminic acid | Neu4,5,7,9Ac4 | V |
| 5-N-acetyl-7,8,9-tri-O-acetylneuraminic acid | Neu5,7,8,9Ac4 | V |
| 5-N-acetyl-4,7,8,9-tetra-O-acetylneuraminic acid | Neu4,5,7,8,9Ac5 | V |
| 5-N-acetyl-9-O-lactylneuraminic acid | Neu5Ac9Lt | V |
| 5-N-acetyl-4-O-acetyl-9-O-lactylneuraminic acid | Neu4,5Ac29Lt | V |
| 5-N-acetyl-7-O-acetyl-9-O-lactylneuraminic acid | Neu5,7Ac29Lt | V |
| 5-N-acetyl-8-O-methylneuraminic acid | Neu5Ac8Me | V, E |
| 5-N-acetyl-9-O-acetyl-8-O-methylneuraminic acid | Neu5,9Ac28Me | V, E |
| 5-N-acetyl-8-O-sulfoneuraminic acid | Neu5Ac8S | V, E |
| 5-N-acetyl-4-O-acetyl-8-O-sulfoneuraminic acid | Neu4,5Ac28S | V, E |
| 5-N-acetyl-9-O-phosphoneuraminic acid | Neu5Ac9P | V |
| 5-N-acetyl-2-deoxy-2,3-didehydroneuraminic acid | Neu2en5Ac | V |
| 5-N-acetyl-9-O-acetyl-2-deoxy-2,3-didehydroneuraminic acid | Neu2en5,9Ac2 | V |
| 5-N-acetyl-2-deoxy-2,3-didehydro-9-O-lactylneuraminic acid | Neu2en5Ac9Lt | V |
| 5-N-acetyl-2,7-anhydroneuraminic acid | Neu2,7an5Ac | V |
| 5-N-acetylneuraminic acid 1,7-lactone | Neu5Acl,7lactone | V |
| 5-N-acetyl-9-O-acetylneuraminic acid 1,7-lactone | Neu5,9Ac21,7lactone | V |
| 5-N-acetyl-4,9-di-O-acetylneuraminic acid 1,7-lactone | Neu4,5,9Ac31,7lactone | V |
| 5-N-glycolylneuraminic acid | Neu5Gc | V, Pz, F |
| 4-O-acetyl-5-N-glycolylneuraminic acid | Neu4Ac5Gc | V |
| 7-O-acetyl-5-N-glycolylneuraminic acid | Neu7Ac5Gc | V |
| 8-O-acetyl-5-N-glycolylneuraminic acid | Neu8Ac5Gc | V |
| 9-O-acetyl-5-N-glycolylneuraminic acid | Neu9Ac5Gc | V, E |
| 4,7-di-O-acetyl-5-N-glycolylneuraminic acid | Neu4,7Ac25Gc | V |
| 4,9-di-O-acetyl-5-N-glycolylneuraminic acid | Neu4,9Ac25Gc | V |
| 7,9-di-O-acetyl-5-N-glycolylneuraminic acid | Neu7,9Ac25Gc | V |
| 8,9-di-O-acetyl-5-N-glycolylneuraminic acid | Neu8,9Ac25Gc | V |
| 7,8,9-tri-O-acetyl-5-N-glycolylneuraminic acid | Neu7,8,9Ac35Gc | V |
| 5-N-glycolyl-9-O-lactylneuraminic acid | Neu5Gc9Lt | V |
| 4-O-acetyl-5-N-glycolyl-9-O-lactylneuraminic acid | Neu4Ac5Gc9Lt | V |
| 8-O-acetyl-5-N-glycolyl-9-O-lactylneuraminic acid | Neu8Ac5Gc9Lt | V |
| 4,7-di-O-acetyl-5-N-glycolyl-9-O-lactylneuraminic acid | Neu4,7Ac25Gc9Lt | V |
| 7,8-di-O-acetyl-5-N-glycolyl-9-O-lactylneuraminic acid | Neu7,8Ac25Gc9Lt | V |
| 5-N-glycolyl-8-O-methylneuraminic acid | Neu5Gc8Me | E |
| 9-O-acetyl-5-N-glycolyl-8-O-methylneuraminic acid | Neu9Ac5Gc8Me | E |
| 7,9-di-O-acetyl-5-N-glycolyl-8-O-methylneuraminic acid | Neu7,9Ac25Gc8Me | E |
| 5-N-glycolyl-8-O-sulfoneuraminic acid | Neu5Gc8S | V, E |
| 5-N-glycolyl-9-O-sulfoneuraminic acid | Neu5Gc9S | E |
| 5-N-(O-acetyl)glycolylneuraminic acid | Neu5GcAc | V |
| 5-N-(O-methyl)glycolylneuraminic acid | Neu5GcMe | E |
| 2-deoxy-2,3-didehydro-5-N-glycolylneuraminic acid | Neu2en5Gc | V |
| 9-O-acetyl-2-deoxy-2,3-didehydro-5-N-glycolylneuraminic acid | Neu2en9Ac5Gc | V |
| 2-deoxy-2,3-didehydro-5-N-glycolyl-9-O-lactylneuraminic acid | Neu2en5Gc9Lt | V |
| 2-deoxy-2,3-didehydro-5-N-glycolyl-8-O-methylneuraminic | Neu2en5Gc8Me | E |
| acid | | |
| 2,7-anhydro-5-N-glycolylneuraminic acid | Neu2,7an5Gc | V |
| 2,7-anhydro-5-N-glycolyl-8-O-methylneuraminic acid | Neu2,7an5Gc8Me | E |
| 5-N-glycolylneuraminic acid 1,7-lactone | Neu5Gc1,7lactone | V |
| 2-keto-3-deoxynononic acid | KDN | V, B |
| 5-O-acetyl-2-keto-3-deoxynononic acid | KDN5Ac | V |
| 7-O-acetyl-2-keto-3-deoxynononic acid | KDN7Ac | V |
| 9-O-acetyl-2-keto-3-deoxynononic acid | KDN9Ac | V |
| 4,5-di-O-acetyl-2-keto-3-deoxynononic acid | KDN4,5Ac2 | V |
| 4,7-di-O-acetyl-2-keto-3-deoxynononic acid | KDN4,7Ac2 | V |
| 5,9-di-O-acetyl-2-keto-3-deoxynononic acid | KDN5,9Ac2 | V |
| 7,9-di-O-acetyl-2-keto-3-deoxynononic acid | KDN7,9Ac2 | V |
| 8,9-di-O-acetyl-2-keto-3-deoxynononic acid | KDN8,9Ac2 | V |
| 2-keto-3-deoxy-5-O-methylnononic acid | KDN5Me | B |
| 2-keto-3-deoxy-9-O-phosphonononic acid | KDN9P | V |

Abbreviations used:
V, vertebrates;
E, echinoderms;
Ps, protostomes (insects and molluscs);
Pz, protozoa;
F, fungi;
B, bacteria. Angata and Varki, Chem Rev. 2002, 102; 439-469

11. Isolated Nucleic Acid

Also provided herein is isolated nucleic acid encoding a plant CMP-sialic acid transporter. The nucleic acid can have the sequence set forth in SEQ ID NO:1 or 2. The nucleic acid can hybridize to the nucleic acid sequence set forth in SEQ ID NO:1 or 2 under stringent conditions. The nucleic acid can encode a polypeptide with at least 70%, 75%, 80%, 85%, 90%, 95% identity to the sequence set forth in SEQ ID NO:6 or 7. Said encoded polypeptide can comprise conservative mutations, deletions, substitutions, or additions.

Also provided herein is an isolated nucleic acid encoding a plant sialyltransferase. The nucleic acid can have sequence set forth in SEQ ID NO:3, 4 or 5. The nucleic acid can hybridize to the nucleic acid sequence set forth in SEQ ID NO: 3, 4 or 5 under stringent conditions. The nucleic acid can encode a polypeptide with at least 70%, 75%, 80%, 85%, 90%, 95% identity to the sequence set forth in SEQ ID NO:8, 9, or 10. Said encoded polypeptide can comprise conservative mutations, deletions, substitutions, or additions.

Also provided is a nucleic acid encoding a plant CMP-sialic acid transporter functionally linked to an expression control sequence. Thus, provided is a nucleic acid comprising the nucleic acid sequence set forth in SEQ ID NO:1, 2, 3, 4, or 5, functionally linked to an expression control sequence.

a) Nucleic Acids

There are a variety of molecules disclosed herein that are nucleic acid based, including for example the nucleic acids that encode, for example SEQ ID NOs:1-5, or fragments thereof. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to the sialylating enzymes disclosed herein, for example SEQ ID NOs:1-5. The sequences for the human analogs of these genes, as well as other anlogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

(3) Primers and Probes

Disclosed are compositions including primers and probes, which are capable of interacting with the disclosed nucleic acids, such as the SEQ ID NOs:1-5 as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the gene typically will be used to produce an amplified DNA product that contains a region of the gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

(4) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the disclosed nucleic acids, such as SEQ ID NOs:1-5, and the nucleic acids used for the generation of SEQ ID NOs:6-10, or the genomic DNA of any of the disclosed nucleic acids, or they can interact with the polypeptide encoded by any of the disclosed nucleic acids, such as SEQ ID NOs:1-5, and the nucleic acids used for the generation of SEQ ID NOs:6-10. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

(5) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

It is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

b) Peptides

As disclosed herein there are numerous variants of the sialylating protein that are herein contemplated. In addition, to the known functional species variants, there are derivatives of the sialylating proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 2 and 3 and are referred to as conservative substitutions.

TABLE 3
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 4
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

c) Sequence Similarities

It is understood that one way to define the variants and derivatives of the disclosed proteins herein is through defining the variants and derivatives in terms of homology/identity to specific known sequences. For example, SEQ ID NO:6 sets forth a particular sequence of CMP-SA transporter and SEQ ID NO:8 sets forth a particular sequence of a sialyltransferase protein. Specifically disclosed are variants of these and other proteins herein disclosed which have at least, 40%, 45%, 50%, 55%, 60%, 65%, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:6 is set forth in SEQ ID NO:1. Another nucleic acid sequence that encodes the same protein sequence set forth in SEQ ID NO: 6 is set forth in SEQ ID NO:11. In addition, for example, a disclosed conservative derivative of SEQ ID NO:6 is shown in SEQ ID NO:12, where the isoleucine (I) at position 15 is changed to a valine (V). It is understood that for this mutation all of the nucleic acid sequences that encode this particular derivative of the CMP-SA transporter are also disclosed including for example SEQ ID NO:13 and SEQ ID NO:14 which set forth two of the degenerate nucleic acid sequences that encode the particular polypeptide set forth in SEQ ID NO:12. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 3 and Table 4. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—);

Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

12. Nucleic Acid Delivery

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

(1) Nucleic Acid Based Delivery Systems

Provided herein is a vector comprising any of the nucleic acids provided herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as SEQ ID NOs:1-5 into the cell without degradation and include a promoter yielding expression of the gene in the cells into which it is delivered. In some embodiments the promoters are derived from either a virus or a retrovirus. Viral vectors include, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Baculovirus

Baculoviruses are widely used for foreign gene expression in insect cells (see, e.g., Smith, et al., U.S. Pat. No. 4,745,051 (recombinant baculovirus) and U.S. Pat. No. 4,879,236; Summers and Smith. A Manual of Methods for Baculovirus Vectors and Insect Cell Culture Procedures, May 1987, Texas A&M University; O'Reilly et al. Baculovirus Expression Vectors A Laboratory Manual, 1994, Oxford University Press; and references therein).

In particular, baculoviruses such as Autographa californica nuclear polyhedrosis virus (AcNPV) are grown in established Lepidoptera insect cell lines including ones derived from ovarian tissue of the fall armyworm (Spodoptera frugiperda) and the cabbage looper (Trichoplusia ni) and midgut tissue from T. ni. The cell lines in most common use to support AcNPV replication and production of recombinant products are S. frugiperda IPLB-SF-21 (Vaughn, et al. In Vitro 13:213-217, 1977) and S. frugiperda Sf-9 cells (Summers and Smith, supra), T. ni TN-368 cells (Hink, Ibid. 1970) and T. ni BTI-TN-5-B1-4 cells (Granados, U.S. Pat. Nos. 5,300,435, 5,298,418).

(b) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(c) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(d) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(e) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed enzymes or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described above, the herein provided recombinant sialylated glyocoproteins can be administered in a pharmaceutically acceptable carrier and can be delivered to a subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

13. Cells

The cell of the herein provided methods can be an Arabidopsis thaliana, Nicotiana tabacum, or Medicago stativa plant cell. As disclosed herein, these cells express endogenous sialylating enzymes at basal levels. It is understood that other plant cells comprise sialylating enzymes. Thus, the cell can be a corn, rice, sugarcane, peanut, tomato, potato, legumes, fruits, tubers, cereal, carrots, sunflower, canola, soybean, cotton, squash, papaya, or lemna cell. Plant cells according to the provided methods can be from a primary cell culture, immortalized cell line, or whole plant or seed.

The cell of the provided method can be a prokaryote. Thus, the cell can be E. coli, or Campylobacter jejuni. The cell of the provided method can be a eukaryote. The cell of the provided method can be a fungal cell, such as yeast cell. The cell can be an insect cell, such as for example an Sf9 or Sf21 cell. The cell of the provided method can be a mammalian cell, such as for example, a human, monkey, murine, rat, bovine, or hamster cell. Thus, the cell can be, for example, a 293, HeLa, MCF-7, WiDr, B16, C2C12, CHO, PC1.0, C6, or COS cell.

Also provided herein is a cell comprising any of the herein provided nucleic acids or vectors. The cell can be any cell that can be transformed with a nucleic acid molecule provided herein. Host cells can be either untransformed cells or cells that are already transformed with at least one nucleic acid molecule (e.g., nucleic acid molecules encoding one or more proteins provided herein). Host cells provided herein either can be endogenously (i.e., naturally) capable of producing the proteins provided herein or can be capable of producing such as a result of engineering, such as by the methods provided herein. Cells provided herein can be any cell capable of producing at least one protein provided herein, and include bacterial, fungal (including yeast), parasite (including helminth, protozoa and ectoparasite), other insect, other animal and plant cells. Thus, the provided cell can be a bacterial, mycobacterial, fungal (e.g., yeast), helminth, insect or mammalian cell.

Thus, provided is a cell comprising a nucleic acid having the sequence set forth in SEQ ID NO:1, 2, 3, 4 or 5. Also provided is a plant cell comprising a nucleic acid having the sequence set forth in SEQ ID NO:1, 2, 3, 4 or 5. Also provided is a mammalian cell comprising a nucleic acid having the sequence set forth in SEQ ID NO:1, 2, 3, 4 or 5. Also provided is a bacterial cell comprising a nucleic acid having the sequence set forth in SEQ ID NO:1, 2, 3, 4 or 5. Also provided is a yeast cell comprising a nucleic acid having the sequence set forth in SEQ ID NO:1, 2, 3, 4 or 5.

Also provided is a cell comprising a nucleic acid encoding a protein having the sequence set forth in SEQ ID NO:6, 7, 8, 9, or 10. Also provided is a plant cell comprising a nucleic acid encoding a protein having the sequence set forth in SEQ ID NO:6, 7, 8, 9, or 10. Also provided is a mammalian cell comprising a nucleic acid encoding a protein having the sequence set forth in SEQ ID NO:6, 7, 8, 9, or 10. Also provided is a bacterial mammalian cell comprising a nucleic acid encoding a protein having the sequence set forth in SEQ ID NO:6, 7, 8, 9, or 10. Also provided is a yeast mammalian cell comprising a nucleic acid encoding a protein having the sequence set forth in SEQ ID NO:6, 7, 8, 9, or 10.

14. Product-by-Process

Provided herein is a sialylated glycoprotein produced by any of the methods disclosed herein. Thus, provided is a sialylated glycoprotein produced by a process comprising administering a nucleic acid encoding the protein to a cell comprising a plant sialylating enzyme. In one aspect, the provided glycoprotein comprises a glycosylation pattern that is different from a glycoprotein produced by methods available in the art. For example, yeast-derived glycoproteins are hypermannosylated compared to mammalian-derived glycoproteins. As another example, CHO cells lack a functional α-2,6 sialyltransferase enzyme, resulting in the exclusive addition of sialic acids to galactose via α-2,3 linkages. In contrast, disclosed herein are plant sialyltransferases capable of α-2,3, α-2,6, and α-2,8 linkages.

The plant sialylating enzyme can be plant CMP-sialic acid transporter. The plant sialylating enzyme can be plant sialyltransferase. Thus, the cell of the method can comprise a nucleic acid encoding plant CMP-sialic acid transporter. The cell of the method can comprise a nucleic acid encoding encoding plant sialyltransferase. The nucleic acid can have the sequence set forth in SEQ ID NO:1, 2, 3, 4, or 5. The nucleic acid can hybridize to SEQ ID NO:1, 2, 3, 4 or 5 under stringent conditions. The nucleic acid can encode a polypeptide with at least 70%, 75%, 80%, 85%, 90%, 95% identity to the sequence set forth in SEQ ID NO:6, 7, 8, 9, or 10. In one aspect, any variation of the sequence is due to a conservative change.

Any cell type can be used in the provided method, including plant, mammalian, bacterial, archae, or fungal (such as for example yeast). Thus, the cell of the method can be an Arabidopsis thaliana, Nicotiana tabacum, or Medicago stativa plant cell. The cell of the method can be overexpressing a plant sialylating enzyme. The cell of the method can comprise a nucleic acid encoding bacterial sialic acid (SA) synthase. The cell of the method can comprise a nucleic acid encoding a mammalian enzyme selected from the group consisting of CMP-SA-synthetase, SA-P-phosphatase, SA-P-synthase, ManNAc-6-kinase, and UDP-GlcNAc-2-epimerase. The cell of the method can comprise a source of UDP-GlcNAc. The cell of the method can comprise molecules with terminal galactose residues.

Examples of sialylated glycoproteins that can be produced include, but are not limited to, activin, adenosine deaminase, angiotensinogen I, antithrombin III, antitrypsin, alpha I, apolipoprotein A-I, apolipoprotein A-II, apolipoprotein C-I, apolipoprotein C-II, polipoprotein C-III, apolipoprotein E, atrial natriuretic factor, chorionic gonadotropin, alpha chain, chorionic gonadotropin, beta chain, chymosin, pro, complement, factor B, complement C2, complement C3, complement C4, complement C9, corticotrophin releasing factor, epidermal growth factor, epidermal growth factor receptor, epoxide dehydratase, erythropoietin esterase inhibitor, C1 factor VIII, factor IX, factor X, fibrinogen, gatrin releasing peptide, glucagons, growth hormone, growth hormone RF, somatocrinin, hemopexin, inhibin, insulin, prepro, insulin-like growth factor I, insulin-like growth factor II, interferon alpha, interferon beta, interferon gamma, interleukin-1, interleukin-2, interluekin-3, kininogen, luteinizing hormone beta subunit, luteinizing hormone releasing hormone, lymphotoxin, mast cell growth factor, nerve growth factor beta subunit, oncogene c-sis, PGDF chain A, pancreatic polypeptide, parathyroid hormone, plasminogen, plasminogen activator, prolactin, proopiomelanocortin, protein C, prothrombin, relaxin, rennin, somatostatin, tachykinin, substances P & K, urokinase, vasoactive intestinal peptide, and vasopressin.

15. Antibodies

Also provided herein is an antibody specific for any of the herein provided polypeptides. Thus, provided is an antibody specific for SEQ ID NO:6, 7, 8, 9, or 10.

(a) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with plant sialylating enzymes. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(b) Administration of Antibodies

Administration of the antibodies can be done as disclosed herein. Nucleic acid approaches for antibody delivery also exist. The antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

16. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

17. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved. Disclosed are computer readable mediums comprising the sequences and information regarding the sequences set forth herein.

18. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include the herein provided cells (e.g., seeds) for producing sialylated glycoproteins, and the reagents for isolating and purifying said proteins.

19. Compositions with Similar Functions

It is understood that the compositions disclosed herein have certain functions, such as sialic acid synthesis, activation, transport, or transfer. Disclosed herein are certain structural requirements for performing the disclosed functions, and it is understood that there are a variety of structures which can perform the same function which are related to the disclosed structures, and that these structures will ultimately achieve the same result.

### C. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:23, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. For example, disclosed are nucleic acids in SEQ ID NOs:1-5. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in in SEQ ID NO:1, 2, 3, 4, or 5 and a sequence controlling the expression of the nucleic acid.

Also disclosed are nucleic acid molecules produced by the process comprising linking in an operative way a nucleic acid molecule comprising a sequence having 80% identity to a sequence set forth in in SEQ ID NO:1, 2, 3, 4, or 5, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth in SEQ ID NO:1, 2, 3, 4, or 5 and a sequence controlling the expression of the nucleic acid.

Disclosed are nucleic acid molecules produced by the process comprising linking in an operative way a nucleic acid molecule comprising a sequence encoding a peptide set forth in in SEQ ID NO:6, 7, 8, 9, or 10, and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NOs: 6, 7, 8, 9, or 10, and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NOs: 6, 7, 8, 9, or 10, wherein any changes are conservative changes, and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are plants produced by the process of transfecting a cell within the plant or seed with any of the nucleic acid molecules disclosed herein.

### D. Methods of Using the Compositions

1. Methods of Using the Compositions as Research Tools

The disclosed compositions can be used in a variety of ways as research tools. For example, the disclosed compositions can be used as either reagents in micro arrays or as reagents to probe or analyze existing microarrays. The disclosed compositions can be used in any known method for isolating or identifying single nucleotide polymorphisms. The compositions can also be used in any method for determining allelic analysis. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Sialoglycoconjugates in Plants

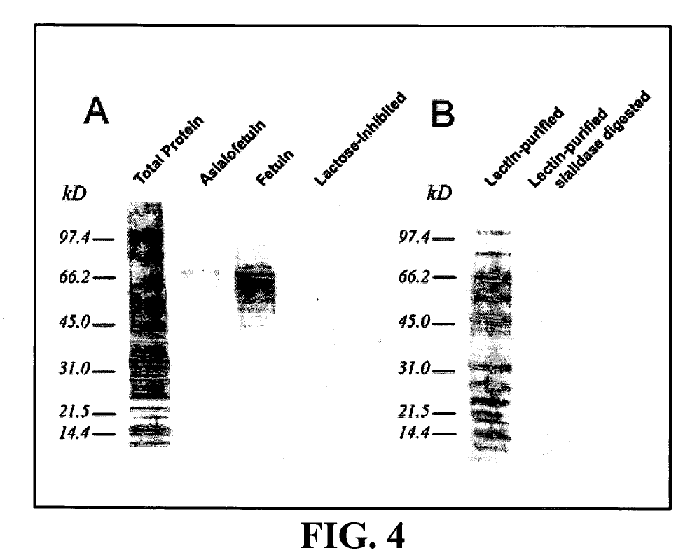

Glycoconjugates from A. thaliana suspension-cultured cells grown on inorganic salts and sucrose were probed with a mixture of biotinylated SNA (Sambucus nigra) and MAA (Maackia amurensis) lectins revealing terminal SAα2,6-Gal and SAα2,3-Gal structures (N. Shibuya et al., Journal of biological chemistry 262, 1596-1601 (1987); R. Cummings, in Guide to techniques in glycobiology G. W. H. Willium J. Lennarz, Ed. (Academic press, San diego, 1994), vol. 230, pp. 66-86). The specificity of lectin binding to sialylated glycoconjugates was verified using fetuin and asialofetuin and inhibition of lectin binding by 100 mM lactose (FIG. 4A).

To further confirm these results, sialylated glycoconjugates were affinity-purified using immobilized SNA and MAA columns, digested with α2-3,6 sialidase from Clostridium perfringens and probed with SNA and MAA lectins. The sialidase-digested proteins did not bind to the lectins, confirming removal of α2-3,6 linked SAs from A. thaliana glycoconjugates (FIG. 4B).

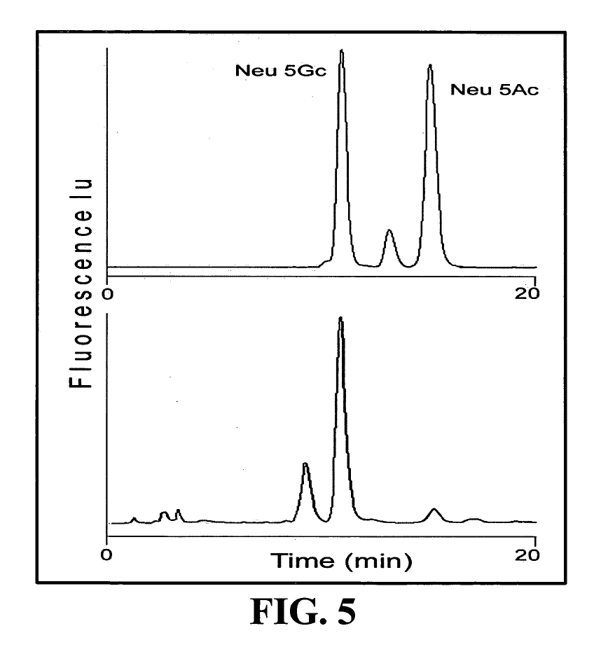

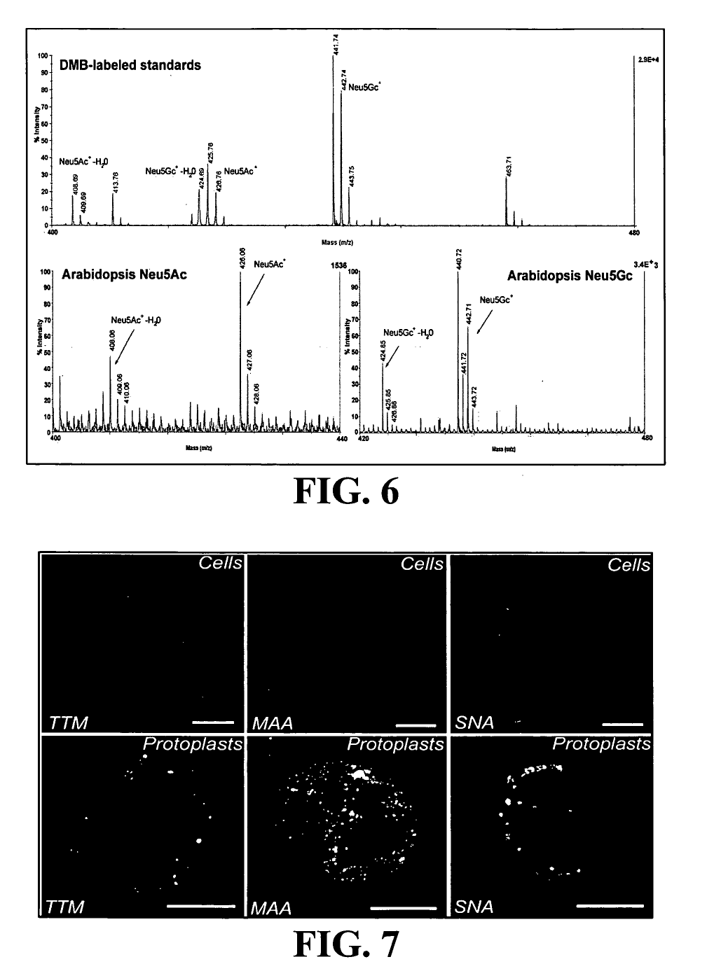

To identify SA species, SAs from A. thaliana proteins were released by mild acid hydrolysis, purified through ion exchange chromatography, labeled with the fluorescent dye 1,2-diamino-4,5-methylene dioxybenzene (DMB) and separated on a reverse-phase C18 column (S. Hara, Y. Takemori, M. Yamaguchi, M. Nakamura, Y. Ohkura, Analytical Biochemistry 164, 138-145 (July, 1987)). Commercially available Neu5Ac and Neu5Gc were used as standards (FIG. 5). In A. thaliana samples, a prominent peak corresponding to Neu5Gc and a smaller peak corresponding to Neu5Ac were detected indicating the presence of these two SAs on A. thaliana glycoconjugates (FIG. 5). Similar results were observed when SAs were removed from glycoconjugates by α2-3,6 sialidase treatment instead of mild acid hydrolysis. Fractions collected from HPLC elution were concentrated and subjected to analysis using LC-ESI and MALDI-TOF. M/z values at 426 and 442 confirm the presence of Neu5Ac and Neu5Gc respectively (FIG. 6).

SA residues (on glycoconjugates) were detected in suspension-cultured A. thaliana cells with the cell wall removed (protoplasts) using biotinylated TTM (Triciticum tritrichomonas), SNA and MAA lectins. The affinity of TTM lectin is for SA regardless of linkage, and it tends to bind more strongly to Neu5Ac (versus Neu5Gc). Cells were treated with pectinase and cellulase to prepare the protoplasts, i.e. to remove cell wall. Anti-biotin antibodies conjugated to Cy2 were used to detect sialylated glycoconjugates using confocal microscopy. SA specific lectins revealed labeled structures on/inside the protoplast (FIG. 7). In a parallel set of experiments with the cell wall left intact, no fluorescence was detected on the cell wall.

### 2. Example 2

### Data Mining of the A. thaliana Genome

The availability of the complete sequence of the A. thaliana genome provides the opportunity to search for orthologs of well-characterized mammalian and microbial SA metabolism genes. Database searches have been successful in yielding sequence information for the last two steps of the biosynthesis of sialoglycoconjugates in A. thaliana catalyzed by CMP-SA-Tr and STs, respectively. Plant orthologs have not been identified to mammalian or bacterial genes encoding any of the various enzymes involved in SA biosynthesis. The biosynthetic pathway of the 8 carbon 3-deoxy-D-manno-octulosonate (KDO, a component of plant rhamnogalacturonan II) in bacteria shares several similarities with that of the 9 carbon SA. The A. thaliana genome possesses putative genes for three steps of the KDO biosynthetic pathway: KDO 8-phosphate synthase (At1g79500 (J. Wu, M. A. Patel, A. K. Sundaram, R. W. Woodard, Biochemical Journal 381, 185-193 (Jul. 1, 2004)), & At1g16340), CMP-KDO synthetase (At1g53000) and KDO transferases (At5g03770). Although ST genes share some sequence similarities with KDOTs, a phylogenetic tree constructed with known STs and KDOTs revealed that KDOTs and STs form distinct branches.

### 3. Example 3

### Functional Characterization of A. thaliana Sialyltransferases

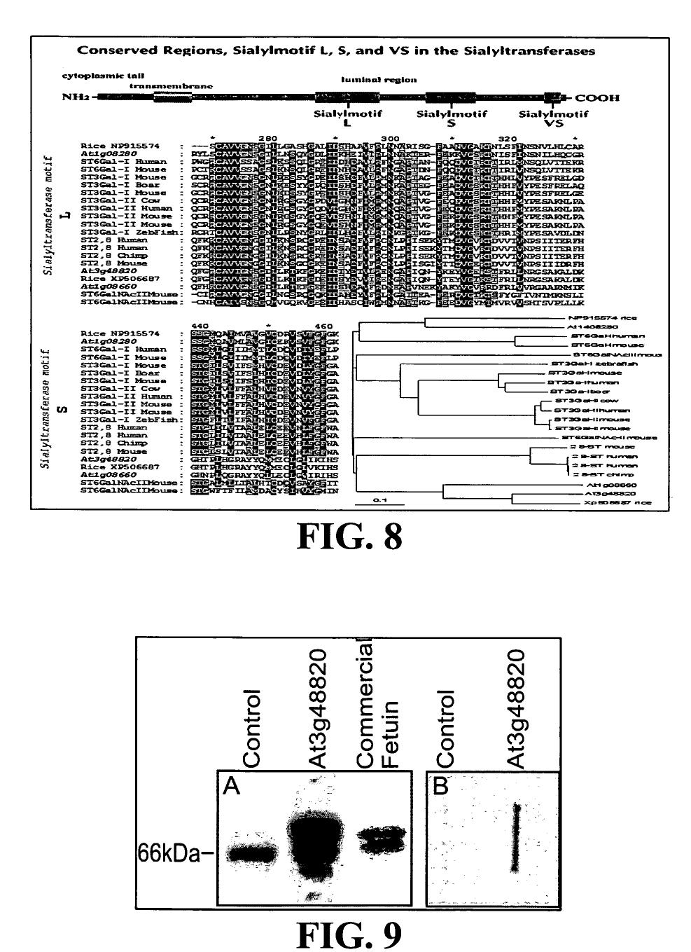

In silico analysis of A. thaliana genes: A search of the A. thaliana genome database at MIPS (http://mips.gsf.de/proj/thal/db/) yielded three sequences (At3g48820, At1g08660 and At1g08280) that belong to the glycosyltransferase family (S. Tsuji, A. K. Datta, J. C. Paulson, glycobiology 6, 5-7 (1996)). This family consists of sialyltransferase (ST) enzymes that can use CMP-N-acetylneuraminate as the sugar donor and catalyze the transfer of SA residues to terminal non-reducing portions of oligosaccharides of glycoproteins and glycolipids. In mammals, STs have been localized to the Golgi complex. STs are type II membrane proteins with a short NH₂-terminal cytoplasmic tail, a 16-20 amino acid signal anchor, a highly variable stem region (20-100 amino acids) and a large COOH-terminal catalytic domain localized in the Golgi lumen (C. K. Paulson J C, Journal of Biological Chemistry 264, 17615-17618 (1989)). The amino acid sequences of STs show sequence homology in three consensus sequences called sialylmotif L (long), sialylmotif S (short), and sialylmotif VS (very short) (S. Tsuji, A. K. Datta, J. C. Paulson, glycobiology 6, 5-7 (1996)) (FIG. 8). Sialylmotif L is involved in the binding of the sugar donor CMP-SA while sialylmotif S binds to both the donor and acceptor molecules (A. K. Datta, A. Sinha, J. C. Paulson, Journal of Biological Chemistry 273, 9608-9618 (1998)). The precise function of the less conserved sialylmotif VS has not been determined but may be involved in the catalytic process (R. A. Geremia, A. Harduin-Lepers, P. Delannoy, glycobiology 7, 5-7 (1997)).

An alignment of the amino acid sequence of putative A. thaliana and rice orthologs of STs with well-characterized mammalian STs shows that sialylmotif L and S are better conserved in plants than the VS motif. The sequence of At3g48820 and At1g08660 appear to be more similar to 2,8 STs while At1g08280 has higher homology to 2,6 type STs (FIG. 8).

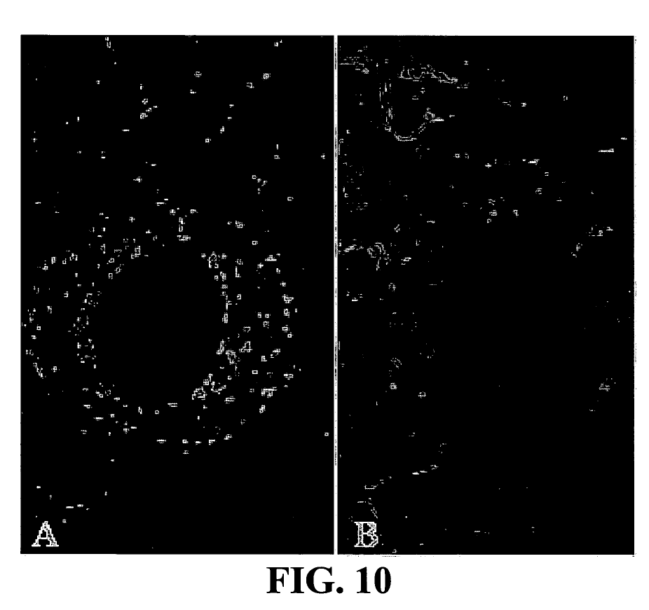

In planta expression and enzyme activity assay of an A. thaliana ST gene: The cDNA for At3g48820, encoding one of the putative A. thaliana STs, was purchased from RIKEN BioResource Center (Ibaraki, Japan). The coding region of the cDNA was PCR-amplified and subcloned into a plant binary expression vector by GATEWAY cloning technology (Invitrogen, CA). Using N. benthamiana leaf discs, Agrobacterium tumefaciens-mediated transformation strategy was employed to generate stably-transformed plants. Microsomal fractions were isolated from leaves of transgenic plants as well as control plants (empty vector transformed) and used in enzyme assays for ST activity with CMP-SA as donor and asialofetuin as acceptor. The reaction mixture was separated by SDS-PAGE, transferred to a PVDF membrane and probed with anti-fetuin antibodies. As shown in FIG. 9A, a significant shift in the mobility of asialofetuin occurred, suggesting sialylation by the recombinant N. benthamiana-expressed A. thaliana ST.

In a similar enzyme assay, the transfer of SA to asialofetuin was further confirmed using CMP-¹⁴C-SA as the donor and asialofetuin as acceptor. The reaction mixture was dot-blotted on to a PVDF membrane and radioactivity transfer to asialofetuin was detected by phosphorimager (Molecular Dynamics Storm 840 system) (FIG. 9B). These two independent enzyme assays indicate that the enzyme encoded by At3g48820 has ST activity.

Subcellular localization of A. thaliana At3g48820 ST: To provide evidence for Golgi localization of A. thaliana At3g48820 ST, the cDNA was C-terminally fused to sGFP coding region, and stably transformed N. benthamiana plants were produced by Agrobacterium-mediated transformation. Leaves from PCR-positive transgenic plants were observed under confocal microscopy after being incubated for 2 hrs in microtubule stabilization buffer only (50 mM PIPES, 5 mM EGTA, 5 mM MgSO₄, pH 6.9) or in buffer, supplemented with 50 μg/ml brefeldin A (BFA), a known inhibitor of ER-Golgi transfer process. The compound changed the typical Golgi-dispersed small punctuate spots pattern (FIG. 10) drastically thus indicating localization of ST-sGFP fusion protein to a BFA-sensitive compartment which is consistent with a Golgi localization.

Complementation of CHO 2A10 cells with A. thaliana ST genes: In order to assess whether the other two A. thaliana genes also code for STs, the cDNA for At1g08280 was cloned by RT-PCR from cDNA prepared from rosette leaves of A. thaliana (var Columbia), while At1g08660 cDNA clone was obtained from Riken BioResource Center. Both the cDNAs were subcloned in a GATEWAY-compatible mammalian expression vector (pDEST40). We used CHO-2A10 cells, deficient in the sialyltransferases ST8SiaIV (M. Windfuhr, A. Manegold, M. Muhlenhoff, M. Eckhardt, R. Gerardy-Schahn, J Biol Chem 275, 32861-70 (Oct. 20, 2000)) (responsible for polysialic acid expression on the cell surface). CHO-2A10 cells were transfected with pDEST40-At1g08280, pDEST40-At1g08660 or pDEST40-GUS (control) plasmids using Lipofectamine 2000 (Invitrogen) according to the manufacturer's instructions. Transfectant lines were selected on 1.4 mg/ml (Invitrogen). Serial passages of stably transfected lines were done in the presence of 1.2 mg/ml geneticin. PCR screening was used to confirm the presence of At1g08660 and At1g08280 transgenes in the transfected and selected CHO-2A10 cells.

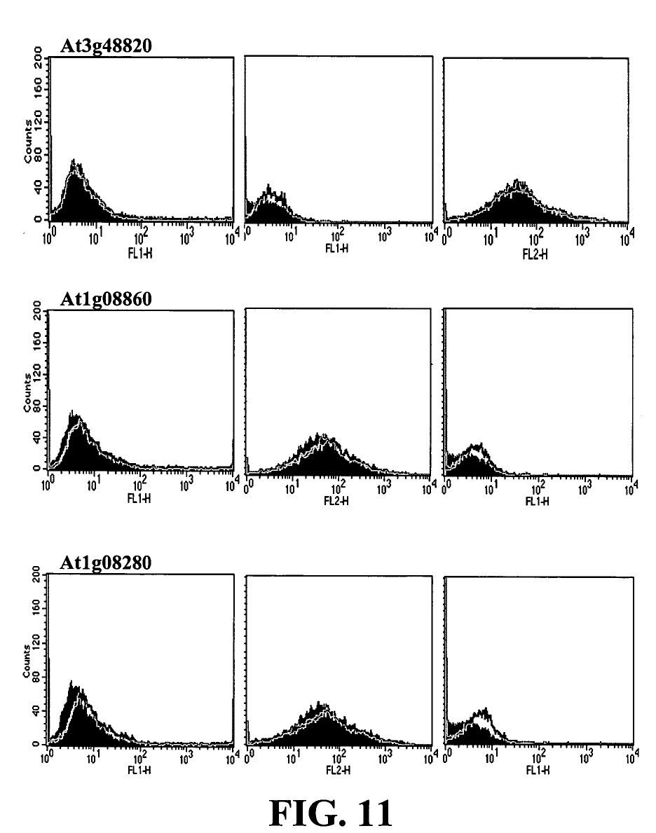

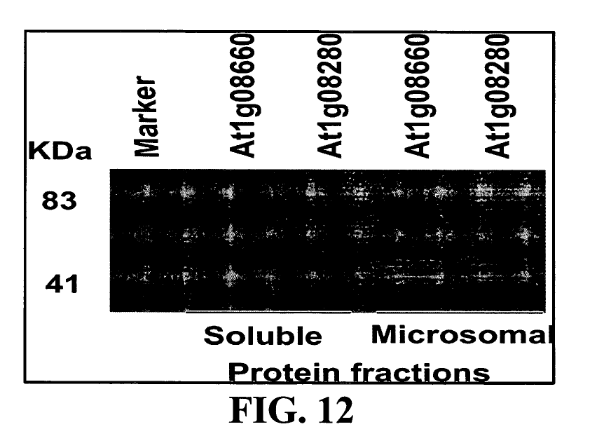

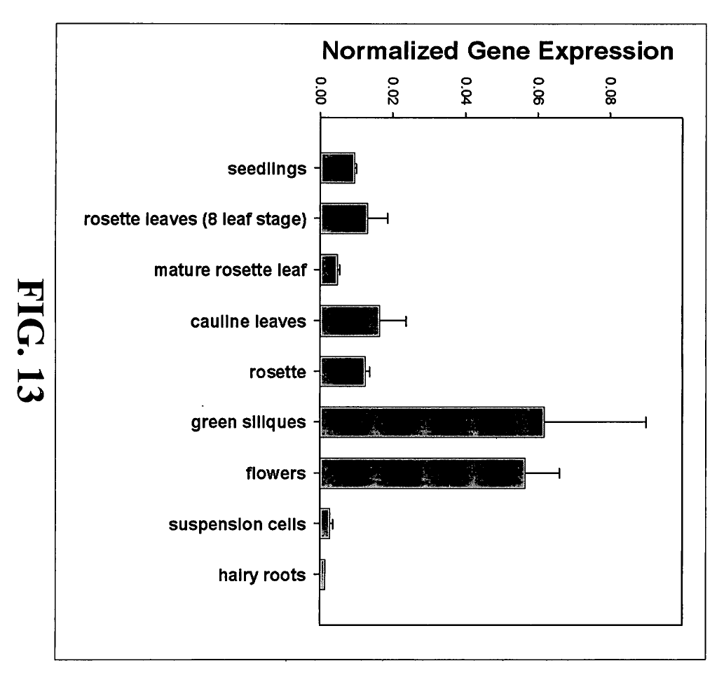

Changes in the cell surface expression of SA, attached by two different linkages, were quantified with FITC labeled SNA and TRITC labeled MAA. Fluorescence-Activated Cell Sorter (FACS) analysis of the lectin stained cells was performed using a FACS instrument (BD Biosciences, MD). CHO-2A10 cells are derived from CHO-K1 cells and lack α2,8 polysialyltransferases. Transfection of these cells with pDEST40-At3g48820 results in transfer of SA in α2,8 linkage. Lack of α2,3 or α2,6 activities suggest that α2,8 may be the primary activity for this enzyme (FIG. 11). Transfection of CHO-2A10 cells with pDEST40-At1g08280 clearly resulted in enhanced expression of α2,8 and α2,6 linked SAs but this gene did not show a significant shift in α2,3 linked SAs (FIG. 11). This indication is also supported by its high similarity to mammalian 2,6 type STs. Cells transfected with pDEST40-At1g08660 show enhanced expression of α2,8 and a marginal increase in both α2,3 and α2,6 linked cell surface SAs, thus indicating a multifunctional but lower ST activity.

Yeast expression of A. thaliana ST genes: S. cerevisiae has been successfully used in the past as a heterologous expression system for the functional characterization of mammalian STs. Both At1g08280 and At1g08660 cDNAs were cloned into the yeast expression vector pDEST-YES52. The stop codon was removed to allow translation of a protein product histidine-tagged (His-tag) at the carboxy terminal. Transformation of S. cerevisiae INVSc-I cells was done using the S.c. Easy Comp Transformation Kit (Invitrogen) and transformants were selected on Yeast Potato Dextrose (YPD) agar plates without uracil. Yeast cells were grown overnight in the presence of galactose (to induce the expression of the transgene), harvested and used for preparation of microsomal fractions. Western blot analysis of the soluble (cytosolic) and microsomal protein fractions with Anti-His antibody clearly shows that the recombinant At1g08280 and At1g08660 proteins are correctly transported to the microsomes (Golgi) of yeast. The absence of any signal from soluble proteins indicates that the subcellular targeting mechanism appears to be highly conserved between yeast and higher plants (FIG. 12). These recombinant proteins are used for biochemical characterization of ST activities.

### 4. Example 4

### Functional Characterization of A. thaliana CMP-SA Transporter

Nucleotide sugars are transported across the ER and Golgi by membrane bound transporter proteins. The A. thaliana genome contains more than 40 members of the Nucleotide Sugar Transporter (NST) family and two of these genes, At5g41760 (SEQ ID NO:1) and At3g59360 (SEQ ID NO:2) were selected based on their similarity to well-characterized mammalian CMP-SA-Trs.

The cDNA for At5g41760 (SEQ ID NO:1) was obtained from Riken Bioresource Center and At3g59360 (SEQ ID NO:2) was PCR-amplified from cDNA prepared from A. thaliana rosette leaves. Mammalian expression constructs were made with these putative transporters (pDEST40-At5g41760 and pDEST40-At3g59360) and evaluated using complementation studies in the Lec2 mutant. Cells transfected with the pDEST40-At5g41760 construct successfully restored CMP-SA transport activity in Lec2 cells, while pDEST40-At3g59360 failed to complement the mutant.

In order to understand the distribution and role of the newly discovered CMP-SA-Tr (designated At-CMP-SA-Trl) in A. thaliana, the tissue specific pattern of endogenous gene expression was examined using real time RT-PCR. Total RNA was extracted from various tissues of A. thaliana, DNAse treated, and used for cDNA preparation. Diluted cDNA was used as a template for real-time RT-PCR using the Applied Biosystems 7900HT system. Copy numbers were calculated from amplification plots of known standards for At-CMP-SA-Trl. Histone transcript levels in the different samples were used to normalize the amounts of At-CMP-SA-Trl. Green siliques and flowers showed higher expression of the transporter gene suggesting a role in reproduction and seed development.

### 5. Example 5

### Profiling of Metabolic Intermediates of the SA Biosynthetic Pathway

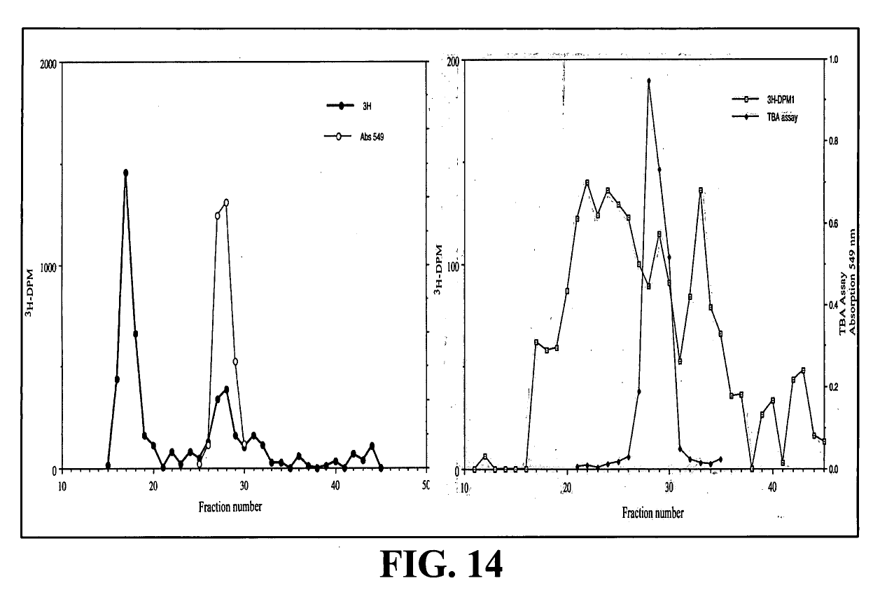

In order to gain knowledge of the nature of the plant SA biosynthetic pathway, two radiolabeled sugars, known to be precursor metabolites in the mammalian SA biosynthetic pathway, were tested. Suspension cultured cells of A. thaliana were grown independently in the presence of ³H-GlcN or ³H-ManNAc (100 microCi each) for 72 hrs. Cells were harvested, separated from the media, and stored at −80° C. A. thaliana media proteins were dialyzed (MWCO 12-14 kDa) and lyophilized. Lyophilized media proteins were reconstituted in PBS, and the radiolabeled secreted glycoproteins partially purified using lectin affinity (Jacalin) and ion exchange (DEAE-cellulose) chromatography. The isolated radiolabeled products were subjected to mild acid hydrolysis to release SAs, the reaction mixture neutralized, mixed with unlabeled NeuSAc as an internal standard and fractionated on Biogel P-2 column. The fractions were monitored for radiolabel by liquid scintillation analysis and SA by the thiobarbituric acid assay (N. Shibuya et al., Journal of biological chemistry 262, 1596-1601 (1987)). As shown in FIG. 14, a fraction of radioactivity fed to the cells as ³H-GlcN or ³H-ManNAc results in labeled SA co-eluting with reference NeuSAc. The peak co-eluting with known Neu5Ac molecule was more prominent in ³H-GlcN fed cells, whereas in the case of ³H-ManNAc fed cells there were other unknown peaks eluting after Neu5Ac, indicating its probable incorporation into as yet unidentified biomolecules.

### 6. Example 6

### Functional Characterization of A. thaliana Sialyltransferases

In planta expression and ST activity of A. thaliana STs: Transgenic N. benthamiana plants expressing At3g48820 (SEQ ID NO:3) have proved be a valuable system to identify the ST activity of this enzyme. The N. benthamiana expression system provides the appropriate post-translational protein modifications and sub-cellular working environment for optimum functioning of A. thaliana STs. Hence, this system can be used to express At1g08660 (SEQ ID NO:4) and At3g48820 (SEQ ID NO:3) ST genes. Golgi isolated from the transgenic plants can be used in similar assays as with the At3g48820 (SEQ ID NO:3) to asses their ability to function as STs. Changes in the native sialoglyconjugates of N. benthamiana due to the expression of A. thaliana STs can be assessed. No phenotypic changes were observed in the transgenic N. benthamiana plants expressing At3g48820 (SEQ ID NO:3), however physiological alterations (abiotic stress responses such as moisture and salinity) can be analyzed. GFP fusion constructs of the A. thaliana STs can be used for sub-cellular localization using confocal microscopy.

Yeast expression of A. thaliana STs: Currently, eukaryotic STs can be divided into four groups, namely ST6Gal, ST6GalNAc, ST3Gal, and ST8Sia, on the basis of the linkages they form and the nature of their sugar acceptor (S. Tsuji, A. K. Datta, J. C. Paulson, glycobiology 6, 5-7 (1996)). Of the three STs selected, the sequence of AT1g08660 (SEQ ID NO:4) and At3g48820 (SEQ ID NO:3) appear to be more similar to α2,8 STs, while At1g08280 (SEQ ID NO:5) groups well with α2,6 type STs (FIG. 8). Although, the tobacco-derived At3g48820 (SEQ ID NO:3) was able to sialylated asialofetuin, it's precise nature of linkage forming activity remains to be elucidated. Endogenous STs in enzyme assays with N. benthamiana expressed A. thaliana STs may interfere with the characterization of specific linkage formations. Therefore, a system is desired that does not contain endogenous ST activity. S. cerevisiae has been used as a heterologous expression system to characterize enzyme properties and sub-cellular localization of STs from humans (P. Mattila et al., Glycobiology 6, 851-859 (December, 1996); C. H. Krezdom et al., European Journal of Biochemistry 220, 809-817 (Mar. 15, 1994)) and it is devoid of any endogenous ST activity. Microsomal localization of C-terminal His-tagged full-length At1g08660 (SEQ ID NO:4) and At1g08280 (SEQ ID NO:5) have been demonstrated in transformed S. cerevisiae (FIG. 11). Localization can be further evaluated for At3g48820 (SEQ ID NO:3). Indirect evidence from CHO 2A10-expressed recombinant AT1g08660 (SEQ ID NO:4) indicates α2,8, α2,6 and 2,3 activities, while At1g08280 (SEQ ID NO:5) appears be capable of predominately α2,8 and α2,6 activity. The yeast expression studies clarify the nature of A. thaliana ST activities. The yeast microsomal fractions can be used for enzyme assays with CMP-SA as donor and a variety of carefully selected commercially available acceptor molecules shown in Table 5 (Glyko, San Leandro, Calif.) in order to determine the various linkage formation specificities. The acceptor molecules can be fluorescently labeled with 2-aminobenzamide (2-AB, Glyko). Changes in the position of elution by HPLC can be determined after assays and compared to labeled standards. Sialylation of asialofetuin can be monitored by a shift on gel electrophoresis and autoradiogram imaging. Subsequently, the kinetic properties of the enzymes can be investigated using the most favored acceptor substrate. Other properties of the enzyme such as pH, temperature dependant activity and inhibitors can be determined. The His-tagged recombinant STs can be purified from yeast microsomal fractions and used for raising antibodies. The anti-A. thaliana-ST-antibodies can be used for localization of the native ST isoforms in plant tissues and detection of the protein in different extracts from A. thaliana.

TABLE 5
Commercially available acceptor molecules.

| | | Possible |
|---|---|---|
| | | linkages for |
| Acceptor molecules | Glycan Structure | sialic acid |
| Core 1 O-glycan | Galβ1-3GalNAc | α2,3; α2,6 |
| Sialyl Core 1 | Neu5Ac2-3Galβ1-3GalNAc | α2,8 |
| Lewis a antigen | Galβ1-3(Fucα1-4)GlcNAc | α2,3 |
| Sialyl Lewis a | Neu5Ac2-3/6Galβ1-3(Fucα1-4)GlcNAc | α2,8 |
| Lewis x antigen | Galβ1-4(Fucα1-3)GlcNAc | α2,3 |
| Sialyl Lewis x | Neu5Ac2-3/6Galβ1-4(Fucα1-3)GlcNAc | α2,8 |
| N-acetyllactosamine | Galβ1-4GlcNAc | α2,3; α2,6 |
| Sialyl-N-acetyllactosamine | Neu5Ac2-3/6Galβ1-4GlcNAc | α2,8 |
| Asialo-, biantennary N-glycan | Galβ1-4GlcNAcβ1-2Manα1-3(Galβ1-4GlcNAcβ1- | α2,3; α2,6 |
| | 2Manα1-6)Manβ1-4GlcNAcβ1-4GlcNAc | |
| Disialo-, biantennary N-glycan | Neu5Ac2-3/6Galβ1-4GlcNAcβ1-2Manα1- | α2,8 |
| | 3(Neu5Ac2-3/6Galβ1-4GlcNAcβ1-2Manα1- | |
| | 6)Manβ1-4GlcNAcβ1-4GlcNAc | |
| Asialo-, biantennary, fucosylated | Galβ1-4GlcNAcβ1-2Manα1-3(Galβ1-4GlcNAcβ1- | α2,3; α2,6 |
| N-glycan | 2Manα1-6)Manβ1-4GlcNAcβ1-4(Fucα1-6)GlcNAc | |
| Disialo-, biantennary, fucosylated | Neu5Ac2-3/6Galβ-4GlcNAcβ1-2Manα1- | α2,8 |
| N-glycan | 3(Neu5Ac2-3/6Galβ1-4GlcNAcβ1-2Manα1- | |
| | 6)Manβ1-4GlcNAcβ1-4(Fucα1-6)GlcNAc | |
| Glycan structures on asialofetuin | | α2,3; α2,6 |

Expression of A. thaliana STs in CHO cells: CHO cells have proved to be instrumental in the identification of several mammalian STs. CHO cells have an advantage as an ST expression system since they have an active SA biosynthetic pathway and CMP-SA can be readily available for transfer to endogenous acceptors by the expressed STs. The CHO-2A10 mutant, which lacks ST8SiaIV activity, showed changes in the binding of linkage specific lectins to the cell surface due to expression of AT1g08660 (SEQ ID NO:4) and At1g08280 (SEQ ID NO:5) genes. This analysis can be extended to the At3g48820 (SEQ ID NO:3) gene. The information from FACS analysis can be followed up with lectin blots and linkage specific sialidase treatment of sialoglyconjugates extracted from transfected cells to identify qualitative and quantitative changes.

### 7. Example 7

### Functional Characterization of A. thaliana SA Transporter

The discovery that A. thaliana indeed possesses a functional CMP-SA-Tr gene is an important step towards unraveling the cellular machinery responsible for assembly of sialoglycoconjugates. S. cerevisiae has been used successfully in the past as a heterologous expression system for newly cloned mammalian CMP-SA-Trs (P. Berninsone, M. Eckhardt, R. GerardySchahn, C. B. Hirschberg, Journal of Biological Chemistry 272, 12616-12619 (May 9, 1997)). In order to exploit this system to further study, the At-CMP-SA-TrlcDNA has been sub-cloned into the yeast expression vector pYES-DEST52. Microsomal fractions isolated from transformed S. cerevisiae expressing At-CMP-SA-Trl can be used for biochemical studies to determine CMP-SA transport kinetics, substrate specificities, temperature and pH dependant activity as well as potential inhibitors. His-tagged At-CMP-SA-Trl produced by yeast can be purified to raise antibodies against At-CMP-SA-Trl. The antibodies can be instrumental in immuno-localization studies.

One interest is the cellular mechanism of regulation of CMP-SA transport into the plant Golgi by A. thaliana CMP-SA-Trl, because studies with other NSTs have revealed multiple nucleotide sugar transport activities (M. Muraoka, M. Kawakita, N. Ishida, Febs Letters 495, 87-93 (APR 20, 2001)). Golgi from yeast cells possesses predominately GDP-Man transport activity with low transport of other nucleotide sugars (P. Berninsone, M. Eckhardt, R. GerardySchahn, C. B. Hirschberg, Journal of Biological Chemistry 272, 12616-12619 (May 9, 1997)) and therefore is a good system to study the transport of various nucleotide sugars. Using isolated Golgi vesicles from S. cerevisiae expressing At-CMP-SA-Trl, the regulation of transport of various commercially available radiolabeled nucleotide sugars such as CMP-SA, UDP-Gal, UDP-Glc, UDP-GlcNAc, UDP-GlcA, UDP-Xyl and GDP-Fuc (individually and in combinations) can be assessed. To assess the transport activity of the endogenous A. thaliana CMP-SA transporter, Golgi isolated from plant cells (D. J. Morre, H. H. Mollenhauer, Journal of Cell Biology 23, 295-& (1964)) can be used. For these studies, seeds of T-DNA mutants of At-CMP-SA-Trl (GABI, Germany) have been obtained and can be used to generate transgenic A. thaliana over-expressing the transporter gene. The mutants, along with transgenics, can be used to assess the changes in the pattern of glycosylation of endogenous glycoconjugates, alterations in growth and development, response to abiotic stresses etc, to understand the physiological role of At-CMP-SA-Trl.

It has been proposed T-DNA mutant and over-expressors of At-CMP-SA-Trl are used to determine if the transport of CMP-SA acts as a control step in the synthesis of sialoglyconjugates (R. Gerardy-Schahn, S. Oelmann, H. Bakker, Biochimie 83, 775-782 (August, 2001)). The feeding of suspension cultured cells with specific sugars can be undertaken to study the impact on the gene expression pattern of At-CMP-SA-Trl to identify probable regulators of gene expression.

### 8. Example 8

### Characterization of Sialoglycoconjugates in A. thaliana

Although many sialoglycoconjugates are known from non-plant species (especially from higher animals), there have hitherto been no reports describing the identity of sialoglycoconjugates in plants. Lectin binding experiments on plant proteins using MAA and SNA have shown that certain proteins show a positive reaction indicating they contain terminal SA residues. However, lectins have limitations in their specificity and lectin binding/blotting is not a quantitative technique. Here, the sialoglycoconjugates present in A. thaliana can be identified using established analytical techniques in addition to lectin binding assays. In other organisms, glycoprotein and glycolipid species have been found to be sialylated. Therefore, extracted plant glycoproteins and glycolipids can be examined for sialylation and structurally characterized as follows:

Detection and identification of sialylated glycoproteins: Proteins can be sequentially extracted from various plant tissues that have been lyophilized and homogenized, and from suspension cultured cells which have been lysed on a French pressure cell press (Thermo Electron Corp.); and resuspended in protease inhibitor containing buffer. Glycoproteins secreted into the media can be recovered by dialyzing against PBS containing protease inhibitors. Plant proteins can be extracted using the ‘Plant Fractionated Protein Extraction Kit’ from Sigma-Aldrich (St. Louise, Mo.). Each of the resulting protein mixtures from cells and the media can be subjected to fractionation by anion exchange chromatography (MonoQ, 50×5 mm column) on an FPLC system (ÄKTA explorer, Amersham Biosciences, NJ). Collected fractions can be desalted and separated by 1-D SDS-PAGE, transferred to PVDF membrane and probed with SA reactive lectins (MAA, SNA-I and LFA—Limus flavus has affinity for SA residues regardless of linkage) to ascertain the peaks containing sialylated glycoproteins. Further purification of the collected fractions of interest can be done by gel-filtration on appropriate matrices and reverse-phase FPLC (C18, Vydac), again with monitoring by 1-D electrophoresis and lectin blotting.

Those fractions shown to react with SA specific lectins can be precipitated by acetone, boiled in SDS sample buffer, and subjected to two-dimensional gel electrophoresis (2-DE). The separated proteins can then be transferred to PVDF membrane and blotted with SA specific lectins to produce a better picture of the sample complexity. Glycoconjugates containing different numbers of SAs can be readily charge-resolved in the first dimension isoelectric focusing of 2-DE.

Putative sialoglycoprotein containing fractions/spots can be subjected to a combined gel-slice nanoLC-MS/MS experiment MudPIT) with trypsin digestion, as described (L. Breci et al., Proteomics 5, 2018-2028 (May, 2005)). The acquired MS/MS spectra can be screened for the presence of diagnostic oxonium fragment ions at m/z 204 and/or 366, corresponding to HexNAc and HexNAc-hexose, respectively (M. J. Huddleston, M. F. Bean, S. A. Carr, Analytical chemistry 65, 877-884 (Apr. 1, 1993)). The presence of a strong signal for either or both of these fragments confirms the presence of a glycoprotein. The proteins identified in silico which are well known and which do not contain any N-linked glycosylation sites or O-linked glycosylation type motifs can be discounted as the source of glycosylation and thus even a few residues of sequence manually interpreted from tandem MS spectra can be used to pinpoint the glycopeptide carrying the PTM (A. Koller et al., Electrophoresis 25, 2003-2009 (July, 2004). The results can then be confirmed by repeating the experiment with different proteases, such as chymotrypsin or high-pH proteinase K (M. J. MacCoss, C. C. Wu, J. R. Yates, Analytical chemistry 74, 5593-5599 (Nov. 1, 2002)), to generate overlapping peptide and glycopeptide maps.

Once the identity of the modified protein involved is known, the sialooligosaccharides present on the glycopeptides can be structurally characterized. PNGase A can be used to release N-linked oligosaccharides, and β-elimination with borohydride reduction to release O-linked oligosaccharides. The liberated oligosaccharides can be cleaned using porous graphitized carbon SPE cartridges and then subjected to structural analysis. Some of the techniques used include nanoLC-MS/MS and direct nano-infusion MSⁿ on an ion trap mass spectrometer of both the native and permethylated oligosaccharides. Derivatization by 2-AB can be used if required, as it is known to generate additional information on branching structures via A and X cross-ring fragmentation, in addition to the more usual Y- and B-ion series (J. Delaney, P. Vouros, Rapid Communications in Mass Spectrometry 15, 325-334 (2001)). Linkage information can be deduced both from the use of exoglycosidase digestions (P. A. Haynes, M. A. Ferguson, G. A. Cross, glycobiology 6, 869-878 (December, 1996)) and the trapping capability of the ion trap instruments, which allows for facile sequential fragmentation along a linear backbone (J. Delaney, P. Vouros, Rapid Commun Mass Spectrom 15, 325-334 (2001)). By using these approaches the primary sequence of the sialooligosaccharides can be deduced without any a priori knowledge of the glycans involved (P. A. Haynes, M. A. Ferguson, G. A. Cross, glycobiology 6, 869-878 (December, 1996)).

Changes in the production of sialoglycoconjugates in mutant plants can be examined by comparing the altered 2-DE profiles with those of wild-type plants and suspension cultured cells. An increase, decrease or disappearance of sialoglycoconjugates can be observed. If proteins have shifted or new proteins have appeared, they can be identified by excising the gel piece of interest, digesting with trypsin and sequencing by nanoLC-MS/MS as described above. The oligosaccharide fractionation and analysis can be repeated for SA Tr and ST mutant plants as described above, to examine whether the genetic changes have any impact at the detailed structural level.

Detection and identification of sialylated glycolipids: Glycolipids can be extracted and examined for the presence of SA as described (M. L. Rodrigues et al., Glycoconjugate Journal 19, 165-173 (2003)). In brief, washed A. thaliana suspension culture cells and other lyophilized plant material (heat treated to inactivate hydrolytic material) can be extracted at room temperature with chloroform/methanol. The crude lipid extract can be fractionated as described (J. Folch, M. Lees, G. H. Sloane Stanley, Journal of Biological Chemistry 226, 497-509 (1957)), and the upper phase examined for SA-containing material by chromatographing on silica gel TLC plates developed with chloroform/methanol/0.02% CaCl₂ and staining for sialyl residues with resorcinol-HCl. The presence of SA can be confirmed by treating the material in the upper phase with mild acid hydrolysis, and analyzing the subsequently released SA by HPAEC-PAD (Dionex, Sunnyvale, Calif.). Additional confirmatory methods for SA identification include derivatization with DMB for HPLC (as per preliminary data) and per-O-acetylation and examination by LC-MS-MS (Waters CapLC QToffII, Waters Corp. MA) with comparison to derivatized commercially purchased standards.

If the results are positive, then fractionation and purification of the individual glycolipids can be carried out by HPLC using a silica column (YMC-Pak SIL-AD, 250×4.6 mm, 5 μm) (T. Sugawara, T. Miyazawa, Lipids 34, 1231-1237 (1999)) with subsequent fraction collection and analyses of fractions by a micro-resorcinol assay for SAs. If further purification is necessary, DEAE-cellulose chromatography can be carried out. The collected fractions can be examined for the presence of SA by TLC with staining as before. Collected purified sialoglycolipid can be structurally characterized by LC-ESI-MS-MS.

### 9. Example 9

### Profiling of Metabolic Intermediates of A. thaliana SA Biosynthesis Pathway

In mammalian and microbial systems, enzymes and intermediates of the SA biosynthetic pathway have been elucidated and identified by a combination of successive chromatographies and precipitations in conjunction with calorimetric assays, enzyme assays, the use of radiolabeled substrates, ion-exchange and paper chromatography, electrophoresis and optical rotation studies (D. G. Comb, S. Roseman (1960); E. L. Kean, S. Roseman (1966); D. R. Watson, et al (1966); V. P. Bhavanandan, et al (1988)). This can be accompanied by the analysis of the products of the reaction by techniques such as mass spectrometry (H. Zhai, P. et al (2005); C. Gerke, et al (1998)) and HPAEC-PAD, capable of detecting picomoles quantities (J. Rohrer, et al (1998)).

Characterization of metabolic intermediates by radiolabeled sugar feeding: In the plant genome databases there is an apparent absence of genetic orthologs to the mammalian enzymes involved in SA biosynthesis. Radiolabeled intermediate sugars have been commonly used to elucidate the components and the regulation of SA biosynthesis (S. Diaz, A. Varki, Analytical chemistry 150, 32-46 (1985); A. Varki, in Methods in Enzymology. (1994), vol. 230, pp. 16-31). As disclosed herein, plant cells do uptake and incorporate tritiated GlcN and ManNAc into SA or related sugars, suggesting that SA biosynthesis pathway in plants has some commonality with the pathways described in other organisms (FIG. 2). This sensitive and biochemically robust technique can be expanded to identify plant Neu5Ac biosynthesis intermediates which, in tandem with the mapping technique detailed below, subsequently identifies the corresponding enzyme activities.

Wild type suspension cultured A. thaliana cells can be fed with radiolabeled GlcN, GlcNAc and ManNAc, and the resultant ‘destination’ sugars can be analyzed by collecting the cells as well as various radiolabeled glycoconjugates secreted into the media. Extracts of the radiolabeled plant cells and the non-dialyzable material in the spent culture media can be fractionated by standard techniques, i.e., gel filtration, ion exchange and reverse-phase chromatography, the fractions in this case screened by liquid scintillation counting. Individual components isolated on the basis of radiolabeled homogeneity can be further characterized. The acid hydrolysates of the radiolabeled glycoconjugates can be analyzed for hexosamines and SAs by gel filtration or HPAEC (collecting fractions and counting—see below). These experiments identify the components of the SA pathway in plants.

Free sugars, sugar phosphates and sugar nucleotides can also be extracted using the same method as below, fractionated on a size-exclusion column with fractions monitored for radiolabel by liquid scintillation analysis and the collected radiolabeled fractions analyzed as detailed above.

Mapping and identification of free sugars, sugar phosphates and sugar nucleotides intermediates: Free sugars can be obtained from suspension cultured cells and plant material by extraction with 80% ethanol and consequent filtration through a 1 kDa cut-off membrane. The extract can be lyophilized and fractionated into neutral and acidic portions by passing through an anion exchange column (AG1-X2, 200-400 mesh, acetate form, Bio-Rad Inc., CA) and eluting successively with 2 mM pyridinium acetate, pH 5.6 and 0.3, 0.5, 2.0 and 4.0 M formic acid, the last in combination with 0.05 M sodium acetate (W. Kundig, S. Ghosh, S. Roseman, Journal of Biological Chemistry 241, 5619-5626 (1966)). Fractions can be collected over ice and, in the case of the acidic eluant, immediately neutralized with pyridine. Media from suspension cultures can also be filtered through a 1 kDa cut-off membrane and free sugars further separated in the same way. The fractions from the ion-exchange column can be lyophilized and analyzed by HPAEC-PAD on a PA10 column (Dionex Corp., Sunnyvale, Calif.) using the monosaccharide, SA and phosphorylated sugar programs. The identity of nucleotides and nucleotide-sugars present can be determined using the HPAEC PA10 column with detection at 260 nm as detailed by (N. Tomiya, E. Ailor, S. M. Lawrence, M. J. Betenbaugh, Y. C. Lee, Analytical Niochemistry 293, 129-137 (2001)). For the analysis of the radiolabel components the system can be first calibrated with appropriate unlabeled and radiolabeled reference standards. For example, CMP-Neu5Ac and CMP-14C-Neu5Ac with detection of unlabeled nucleotide sugar by measuring absorbance at 260 nm and detection of the radiolabel nucleotide sugar by collection of one minute fractions and scintillation counting. The unknown radiolabeled sample can be mixed with an internal reference and analyzed as above. The identity of sugar phosphates and nucleotide-sugars can be confirmed by co-elution of the unknown and the reference standard on rechromatography.

This method is especially useful in comparing the changes in metabolic intermediates produced by wild-type, SA Tr and ST mutants and suspension cultures by chromatogram mapping as above. The increased or new peak fractions can be collected, derivatized and analyzed by HPLC or LC-MS-MS.

### F. REFERENCES

R. Schauer, Trends in Biochemical Sciences 10, 357-360 (1985).
S. Kelm, R. Schauer, in International Review of Cytology K. W. Jeon, J. W. Jarvik, Eds. (Academic Press, San Diego, 1997) pp. 137-140.
R. Schauer, Glycoconjugate Journal 17, 485-499 (2000).
M. M. Shah, K. Fujiyama, C. R. Flynn, L. Joshi, Nat Biotechnol 21, 1470-1 (December, 2003).
A. Varki, in Essentials of Glycobiology J. E. Ajit Varki R C, Hudson Freeze, Gerald Hart, Jamey Marth, Ed. (Cold Spring Harbor Laboratory Press, New York, 1999) pp. 85-100.
A. Varki, in Essentials of Glycobiology J. E. Ajit Varki, Hudson Freeze, Gerald Hart, Jamey Marth, Ed. (Cold Spring Harbor Laboratory Press, New York, 1999) pp. 85-100.
L. Joshi, M. L. Shuler, H. A. Wood, Biotechnology Progress 17, 822-827 (September-October, 2001).
M. L. Rodrigues et al., Glycoconjugate Journal 19, 165-173 (2003).
T. Angata, A. Varki, Chemical Reviews 102, 439-469 (February, 2002).
L. Shaw, R. Schauer, Biological Chemistry Hoppe-Seyler 369, 477-486 (1988).
M. Gollub, R. Schauer, L. Shaw, Comparative Biochemistry and Physiology Part B 120, 605-615 (1998).
S. R. Reutter W, Stehling P, Baum O., in Glycosciences-Status and Perspectives. H.-J. Gabius, S. Gabius, Eds. (Chapman Hall, Weinheim, 1997) pp. 245-259.
O. T. Keppler et al., Science 284, 1372-1376 (1999).
S. M. Lawrence et al., Journal of Biological Chemistry 275, 17869-17877 (Jun. 9, 2000).
A. P. Corfield, R. Schauer, M. Wember, Biochemistry 177, 1-7 (1979).
R. Gerardy-Schahn, S. Oelmann, H. Bakker, Biochimie 83, 775-782 (Aug. 2001).
S. Oelmann, P. Stanley, R. Gerardy-Schahn, Journal of Biological Chemistry 276, 26291-26300 (2001).
N. Taniguchi, Honke K, Fukuda M., Handbook of glycosyltransferases and related genes. (Springer-Verlag, Tokyo, 2002).
I. B. Wilson et al., glycobiology 11, 261-274 (2001).
A. Varki, in Essentials of glycobiology J. E. Ajit Varki R C, Hudson Freeze, Gerald Hart, Jamey Marth, Ed. (Cold spring harbor laboratory press, New York, 1999) pp. 101-113.
T. Kishimoto, M. Watanabe, T. Mitsui, H. Hori, Archives of Biochemistry and Biophysics (1999), vol. 370.
K. Fujiyama, L. Joshi, glycobiology 13, 871-871 (NOV, 2003).
V. P. Bhavanandan, K. Furukawa, Biochemistry and Oncology of Sialoglycoprotens. A. Rosenberg, Ed., Biology of Sialic acids (Plenum Press, New York, 1995).
N. Shibuya et al., Journal of biological chemistry 262, 1596-1601 (1987).
R. Cummings, in Guide to techniques in glycobiology G. W. H. Willium J. Lennarz, Ed. (Academic press, San diego, 1994), vol. 230, pp. 66-86.
S. Hara, Y. Takemori, M. Yamaguchi, M. Nakamura, Y. Ohkura, Analytical Biochemistry 164, 138-145 (July, 1987).
C. K. Paulson J C, Journal of Biological Chemistry 264, 17615-17618 (1989).
S. Tsuji, A. K. Datta, J. C. Paulson, glycobiology 6, 5-7 (1996).
A. K. Datta, A. Sinha, J. C. Paulson, Journal of Biological Chemistry 273, 9608-9618 (1998).
R. A. Geremia, A. Harduin-Lepers, P. Delannoy, glycobiology 7, 5-7 (1997).
M. Windfuhr, A. Manegold, M. Muhlenhoff, M. Eckhardt, R. Gerardy-Schahn, J Biol Chem 275, 32861-70 (Oct. 20, 2000).
M. Eckhardt, M. Muhlenhoff, A. Bethe, R. Gerardy-Schahn, Proceedings og national academy of sciences USA 93, 7572-7576 (1996).
H. Bakker et al., glycobiology 15, 193-201 (February, 2005).
A. K. Yeh, D. R. Tulsiani, R. Carubelli, Journal of Laboratory and Clinical Medicine 78, 771-778 (1971).
J. Wu, M. A. Patel, A. K. Sundaram, R. W. Woodard, Biochemical Journal 381, 185-193 (Jul. 1, 2004).
P. Mattila et al., Glycobiology 6, 851-859 (December, 1996).
C. H. Krezdorn et al., European Journal of Biochemistry 220, 809-817 (Mar. 15, 1994).
P. Berninsone, M. Eckhardt, R. GerardySchahn, C. B. Hirschberg, Journal of Biological Chemistry 272, 12616-12619 (May 9, 1997).
M. Muraoka, M. Kawakita, N. Ishida, Febs Letters 495, 87-93 (Apr. 20, 2001).
D. J. Morre, H. H. Mollenhauer, Journal of Cell Biology 23, 295-& (1964).
L. Breci et al., Proteomics 5, 2018-2028 (May, 2005).
M. J. Huddleston, M. F. Bean, S. A. Carr, Analytical chemistry 65, 877-884 (Apr. 1, 1993).
A. Koller et al., Electrophoresis 25, 2003-2009 (Jul., 2004).
M. J. MacCoss, C. C. Wu, J. R. Yates, Analytical chemistry 74, 5593-5599 (Nov. 1, 2002).
J. Delaney, P. Vouros, Rapid Communications in Mass Spectrometry 15, 325-334 (2001).
P. A. Haynes, M. A. Ferguson, G. A. Cross, glycobiology 6, 869-878 (December, 1996).
J. Delaney, P. Vouros, Rapid Commun Mass Spectrom 15, 325-334 (2001).
J. Folch, M. Lees, G. H. Sloane Stanley, Journal of Biological Chemistry 226, 497-509 (1957).
T. Sugawara, T. Miyazawa, Lipids 34, 1231-1237 (1999).
D. G. Comb, S. Roseman, Journal of Biological Chemistry 235, 2529-2537 (1960).
E. L. Kean, S. Roseman, Journal of Biological Chemistry 241, 5643-5650 (1966).
D. R. Watson, G. W. Jourdian, S. Roseman, Journal of Biological Chemistry 241, 5627-5636 (1966).
V. P. Bhavanandan, M. Murrey, E. A. Davidson, Glycoconjugate Journal 5, 467-484 (1988).
H. Zhai, P. C. Dorrestein, A. Chatterjee, T. P. Begley, F. W. McLafferty, Journal of the American Society for Mass Spectrometry 16, 1052-1059 (2005).
C. Gerke, A. Kraft, R. Süβmuth, O. Schweitzer, Friedrich Gotz, Journal of Biological Chemistry 273, 18586-18593 (1998).
J. Rohrer, J. Thayer, M. Weitzhandler, N. Avalovic, glycobiology 8, 35-45 (1998).
S. Diaz, A. Varki, Analytical chemistry 150, 32-46 (1985).
A. Varki, in Methods in Enzymology. (1994), vol. 230, pp. 16-31.
W. Kundig, S. Ghosh, S. Roseman, Journal of Biological Chemistry 241, 5619-5626 (1966).
N. Tomiya, E. Ailor, S. M. Lawrence, M. J. Betenbaugh, Y. C. Lee, Analytical Niochemistry 293, 129-137 (2001).

What is claimed is:

1. A method for producing recombinant sialylated glycoproteins, comprising administering a nucleic acid encoding the protein to a cell comprising a plant sialylating enzyme.

2. The method of claim 1, wherein the plant sialylating enzyme is CMP-sialic acid transporter.

3. The method of claim 2, wherein the cell comprises a nucleic acid encoding plant CMP-sialic acid transporter.

4. The method of claim 3, wherein the nucleic acid has sequence set forth in SEQ ID NO:1 or 2.

5. The method of claim 3, where the nucleic acid hybridizes to SEQ ID NO:1 or 2 under stringent conditions.

6. The method of claim 3, wherein the nucleic acid encodes a polypeptide with at least 70%, 75%, 80%, 85%, 90%, 95% identity to the sequence set forth in SEQ ID NO:6 or 7.

7. The method of claim 6, wherein any change is a conservative change.

8. The method of claim 1, wherein the plant sialylating enzyme is plant sialyltransferase.

9. The method of claim 9, wherein the cell comprises a nucleic acid encoding plant sialyltransferase.

10. The method of claim 9, wherein the nucleic acid has sequence set forth in SEQ ID NO:3, 4 or 5.

11. The method of claim 9, where the nucleic acid hybridizes to SEQ ID NO: 3, 4 or 5 under stringent conditions.

12. The method of claim 9, wherein the nucleic acid encodes a polypeptide with at least 70%, 75%, 80%, 85%, 90%, 95% identity to the sequence set forth in SEQ ID NO:8, 9, or 10.

13. The method of claim 11, wherein any change is a conservative change.

14. The method of claim 1, wherein the cell is an Arabidopsis thaliana, Nicotiana tabacum, or Medicago stativa plant cell.

15. The method of claim 1, wherein the cell is overexpressing a plant sialylating enzyme.

16. The method of claim 1, wherein the cell comprises a nucleic acid encoding bacterial sialic acid (SA) synthase.

17. The method of claim 1, wherein the cell comprises a nucleic acid encoding a mammalian enzyme selected from the group consisting of CMP-SA-synthetase, SA-P-phosphatase, SA-P-synthase, ManNAc-6-kinase, and UDP-GlcNAc-2-epimerase.

18. The method of claim 1, wherein the cell comprises a source of UDP-GlcNAc.

19. The method of claim 1, wherein the protein is selected from the group consisting of activin, adenosine deaminase, angiotensinogen I, antithrombin III, antitrypsin, alpha I, apolipoprotein A-I, apolipoprotein A-II, apolipoprotein C-I, apolipoprotein C-II, polipoprotein C-III, apolipoprotein E, atrial natriuretic factor, chorionic gonadotropin, alpha chain, chorionic gonadotropin, beta chain, chymosin, pro, complement, factor B, complement C2, complement C3, complement C4, complement C9, corticotrophin releasing factor, epidermal growth factor, epidermal growth factor receptor, epoxide dehydratase, erythropoietin esterase inhibitor, C1 factor VIII, factor IX, factor X, fibrinogen, gatrin releasing peptide, glucagons, growth hormone, growth hormone RF, somatocrinin, hemopexin, inhibin, insulin, prepro, insulin-like growth factor I, insulin-like growth factor II, interferon alpha, interferon beta, interferon gamma, interleukin-1, interleukin-2, interluekin-3, kininogen, luteinizing hormone beta subunit, luteinizing hormone releasing hormone, lymphotoxin, mast cell growth factor, nerve growth factor beta subunit, oncogene c-sis, PGDF chain A, pancreatic polypeptide, parathyroid hormone, plasminogen, plasminogen activator, prolactin, proopiomelanocortin, protein C, prothrombin, relaxin, rennin, somatostatin, tachykinin, substances P & K, urokinase, vasoactive intestinal peptide, vasopressin, immunoglobulin, vaccine epitope, neurotrophic factor, and a hormone.

20. An isolated nucleic acid encoding plant CMP-sialic acid transporter.

21. The nucleic acid of claim 20, wherein the nucleic acid has sequence set forth in SEQ ID NO:1 or 2.

22. The nucleic acid of claim 20, wherein the nucleic acid hybridizes to SEQ ID NO:1 or 2 under stringent conditions.

23. The nucleic acid of claim 20, wherein the nucleic acid encodes a polypeptide with at least 70%, 75%, 80%, 85%, 90%, 95% identity to the sequence set forth in SEQ ID NO:6 or 7.

24. The method of claim 23, wherein any change is a conservative change.

25. An isolated nucleic acid encoding plant sialyltransferase.

26. The nucleic acid of claim 25, wherein the nucleic acid has sequence set forth in SEQ ID NO:3, 4 or 5.

27. The nucleic acid of claim 25, wherein the nucleic acid hybridizes to SEQ ID NO: 3, 4 or 5 under stringent conditions.

28. The nucleic acid of claim 25, wherein the nucleic acid encodes a polypeptide with at least 70%, 75%, 80%, 85%, 90%, 95% identity to the sequence set forth in SEQ ID NO:8, 9, or 10.

29. The method of claim 28, wherein any change is a conservative change.

30. A vector comprising the nucleic acid of claim 20 or 25.

31. A cell comprising the nucleic acid of claim 20 or 25.

32. The cell of claim 31, wherein the cell is an Arabidopsis thaliana, Nicotiana tabacum, or Medicago stativa plant cell.

33. The cell of claim 31, wherein the cell is a human, murine, bacterial, archeal, or fungal cell.

34. A method for engineering plants to produce recombinant sialylated glycoproteins, comprising administering to the cell a nucleic acid encoding a plant sialylating enzyme.

35. The method of claim 34, wherein the plant sialylating enzyme is CMP-sialic acid transporter.

36. The method of claim 35, wherein the nucleic acid has sequence SEQ ID NO:1 or 2.

37. The method of claim 34, wherein the plant sialylating enzyme is sialyltransferase.

38. The method of claim 37, wherein the nucleic acid has sequence SEQ ID NO:3, 4 or 5.

39. The method of claim 34, wherein the plant cell is an Arabidopsis thaliana, Nicotiana tabacum, or Medicago stativa plant cell.

40. The method of claim 34, further comprising administering to the plant cell a nucleic acid encoding a mammalian enzyme selected from the group consisting of CMP-SA-synthetase, SA-P-phosphatase, SA-P-synthase, ManNAc-6-kinase, and UDP-GlcNAc-2-epimerase.

41. A sialylated glycoprotein produced by the method of claim 1.

42. The method of claim 41, wherein the protein is selected from the group consisting of activin, adenosine deaminase, angiotensinogen I, antithrombin III, antitrypsin, alpha I, apolipoprotein A-I, apolipoprotein A-II, apolipoprotein C-I, apolipoprotein C-II, polipoprotein C-III, apolipoprotein E, atrial natriuretic factor, chorionic gonadotropin, alpha chain, chorionic gonadotropin, beta chain, chymosin, pro, complement, factor B, complement C2, complement C3, complement C4, complement C9, corticotrophin releasing factor, epidermal growth factor, epidermal growth factor receptor, epoxide dehydratase, erythropoietin esterase inhibitor, C1 factor VIII, factor IX, factor X, fibrinogen, gatrin releasing peptide, glucagons, growth hormone, growth hormone RF, somatocrinin, hemopexin, inhibin, insulin, prepro, insulin-like growth factor I, insulin-like growth factor II, interferon alpha, interferon beta, interferon gamma, interleukin-1, interleukin-2, interluekin-3, kininogen, luteinizing hormone beta subunit, luteinizing hormone releasing hormone, lymphotoxin, mast cell growth factor, nerve growth factor beta subunit, oncogene c-sis, PGDF chain A, pancreatic polypeptide, parathyroid hormone, plasminogen, plasminogen activator, prolactin, proopiomelanocortin, protein C, prothrombin, relaxin, rennin, somatostatin, tachykinin, substances P & K, urokinase, vasoactive intestinal peptide, vasopressin, immunoglobulin, vaccine epitope, neurotrophic factor, and a hormone.


SEQUENCE LISTING (32 sequences)

SEQ ID NO: 1 - DNA, 1362 bp, Artificial Sequence
  Description of Artificial Sequence; note = synthetic construct
ttggtgagaagagagactgaagaagagctacaaaaaatgagagagctcctgtgaagtgacgaagagacgaagaagaaagagaacattaatggcggctactccgtggtactttgtcgcagtacttctcaccattcttaccagctctcaggggattctaacaactctttctcaaagtgatggaggttataagtatgattatgctactgttccctttcttgctgaagtttttaagctgatcatttctggtttatttctctggagagagatgcggacatcttcctcaacaacttcaaggattactacggattggaagagtgtgcgattatttgttattccgtctcttatttatctgatacataacaatgtgcagtttgctacattgacttatgtggatacgtccacttatcagataatgggtaatttgaagattgtgaccactggcatactgtttaggttatttttgaaaaggaaactatctaaacttcagtggatggcaattggtcttttagctgttggaacaactaccagccaggttaagggttgtggagaagcttcttgtgattctttattcacggcaccaatacaaggttatttgctgggcatcctctcagctggtttgtccgcgctagctggaatctacacagagtttctgatgaagagaaacaatgacaccttatactggcaaaacttgcagttatatacgtttggttcacttttcaacgtagctcgacttatagcagatgatttcagacacgggtttgaaaagggtccttggtggcaacgtatctttgatgggtacagcatcaccacttggctggtagttctgaatctcggatccactggcttactggtctcgtggttaatgaagtatgcagacaacatcgtcaaggtttattctacatcaatggcgatgctacttactatggttgcatccatatacctttttagcttcaaaccgacactgcagcttttcttgggtattgtcatatgcatcatgtcactccatatgtactttgctcctccacacacgctcgtggacttgcccgtgacaaacgaagcacatgccaagaccttaaagcaagttgtcgtagaagaaaagacagattcttgaattaaattggacgaaaaggttagttgaacacatagaaaacaatttgctaagtaaatgaacacaatgatcttcttctagcttcatttctattagagaatgagaaacttggatgtgagttcttttgaagaaatgatctgatagtttgtttattcacgtaccgattcttttctttgtaaatgtacatcgaggtgcctagaaatagtaaattcgattgttgaaaaaaaaaaaaaaaaaaaaaaaaaaaaaaaaaa

SEQ ID NO: 2 - DNA, 1865 bp, Artificial Sequence
  Description of Artificial Sequence; note = synthetic construct
aaaaaaagagagaaggagaggtcgtgttagataattagagcgcacttgcgagcaacaacgaacgacgacccactgattcaatctcctcctctctgcatattccttggcatcttttctggtaccgaagctgagtctctggagagtttctagatcttcttcaattctgcaattcagagcgaagatttattagtgattgattattcactctggagctcgagttctggatccgtgtggctggagggaatgttctgttaagctgattgtttaggcttgttggatcagtatagaaattgtcagattgagaaagacaacaaatgaagaacggtatagctgagtgccctgcatgtcattcaaaattggtctctcctggtagcaaaactatttctagggcttatgacgaccacaagattagggtatcctcaaaacaacgtgtcctcaatgtccttttggttgtaggtgattgcatgcttgttggtctccagcctgtcttggtgtatatgtcgaaggtggatgggaaattcaacttcagtcctattagcgtcaacttcttgacagaaatcgcaaaagttatttttgcgattgtgatgcttttaatccaggccaggcaccaaaaagttggagagaagcctcttctatccgtttccacatttgtccaggcagcccgaaataatgtgcttcttgctgttccagccctgttgtatgcaattaataactatctgaagttcacaatgcagctatatttcaatcctgctactgtaaagatgcttagcaacctaaaggttctggtcatcgctgttctattaaagatggtaatgaagcggcggttttctatcatacagtgggaggcacttgctctgttgctgatagggattagtgtaaaccaattacgttctcttcctgaaggtgctacagccattggaattcctcttgctacgggtgcatacgtctgcaccgttatctttgtcactgtcccgtcaatggcatctgtcttcaacgagtatgctctaaagagtcagtatgacacaagcatatatcttcagaatttattcctctatggttatggtgcaatattcaactttcttggaatactaggaactgttatatataaaggtcctggcagctttgacatcctacaaggacattctagggctaccatgtttttgatactgaacaacgcagcacaagggattttatcgtcctttttcttcaaatatgcagatacaatcttgaagaaatattcatcgacagttgccacaatatttactggcatagcatctgctgcgctctttggacatgttataaccatgaacttccttctcggaatttctatcgttttcatttcgatgcaccagttcttttcaccacttgccaaagcgagagacgagcaacaacagaatgggaacctagaactaggcaacacaaaggatactcacagggctaatgaatcattcatcaacatggccgcaggagcaaatgaagaggctagtcaccgcggtgaatcagacgacaggactccactgcttcccagatgaaaagtttcttaaatgatttttgttcggaagaattattttggccaagaatattctagaaattgggggttttccattcgtatactagcagaggaaacagtgaagcaaacttcatgacaccaactcttacttctctcaagagtcggttttaaagttcgttgaagaacctctcaagtctgaagctgtattgttttaagatagagtgagaacaaaattggtaagtgatatatgtattttggatatataatatttgtattctttggtttttgcaaaatatcaggtcacacttttttgctcttgtttatacaattactctcaccaaaaaaaaaaaaaaaa

SEQ ID NO: 3 - DNA, 1752 bp, Artificial Sequence
  Description of Artificial Sequence; note = synthetic construct
ggaaatcacgaagctgaagcttctactggggaatcgatgaagctcttgcatctgatattcttattagctttgactactggaatctctgctgttctaatctacattatcggcgtctctaatctctacgagtctaatcgttttaccaatgaagatttagaggcgttgcagtctttgcagaacggattccagaagtgcgtaagtgcaaatggtttgggactacaagctgctatgggtagagattattgcaaagtttctataaacttccctaaggatactgtccccaaatggaaagatcccaagtctggagagcttgaaggattgtcatatgagtttgacctgtgtgaagcagtagctacatgggaacaagtcaggaatagctctacaatactcaccaaggaatacattgatgccttaccaaatggatgggaggattatgcatggcgcagaatcaataaaggaattcaacttaatcgttgccagaataaatctttatgcattgagaagctttcattggtgcttcctgaaacaccaccatatttcccccggcagtttgggcgatgtgctgttattggtaactctggtgatctcttaaaaacaaagttcggaaaggagatagatacttatgatacggttctaagagaaaatggtgctcctattcagaactacaaggaatatgtgggagagaaaagtacattccgtcttcttaatagagggtcagcaaaagcccttgacaaagtcgtggagttggatgaaaaaaagcaagaggtcctgctcgtcaaaacaacaatacatgatattatgaacaagatgattcgggaagttccaataaaaaatcctgtgtacttaatgctcggtgcttcatttggctcagcagccaaaggaactgggctcaaggctcttgagtttgctctctcgacgtgtgattcagtggatatgtatggattcacagtggatccaggctataaagagtggacaagatatttttcagaatcccgacaaggacatactcctttgcatggaagagcttactaccagatgatggaatgcttgggtctcattaaaatacactctcccatgagagctgatccaaaccgtgtcgtgaaatgggtgccaagccgcagtacaattagatctgcaagaattgcagcagaaaagctcttaaggagagttggagcaggatctgcagatccattagcttcatgctccatagtaaagaagagaaacaaaaacaagcgtccaatggtctcacacctcagaaaacctgtgagcgaccatcaaaaatttgtgagaagcacgagcatgtacccagtggagcacagtccaggacatggtcagctttgcattacaccagctgactaagaaactgcatatctaggaattgatttatcaccatacacactaaagcacaaaacaggacgacgatacattccttcaaaaacaagtccctctgcttttgcgccattgttaggagttgaagatcatcaaggtgacagttatatgcgtaatctctctaggttaggtttaattcttttgttatctatttttccggaattttcttcaccagagatgtaggtaaaatcatagaggactgaaagagaacaaagctagaaattgattatgtctaattttagtacataagcaaatgttatgttctttgttctcttttcttgcctcctctagtccatgtcttaccttgtactagtcacaaaattatattaagcagaatagtgtaaagcaaaaaaaaaaaaaaaa

SEQ ID NO: 4 - DNA, 1586 bp, Artificial Sequence
  Description of Artificial Sequence; note = synthetic construct
gagtttcttggatcatcacactcacctgcttcgttactcttcccttggattggattggattggattcgctctttcatggttgaagaatgagatctcaccaagctggccgcaaacttccgcttcttcaacttctcggctgcgtcgctgttttctccgttttcgttttcaccatccaatcttccttcttcgcagataataatcgtaaactagatcttcagccggaagatatccagatcttgtcagattttcagtccagcgttcaacagtgcgtggctaacagagggcttggactctctgcccatattatcgatcactgtaatcttatcctcaagttccctgaaggcactaacagtacttggtacaatgcacaatttaaagtttttgaagcattggagttcaagtataatgtttgtgaggcagttctcttatgggagcagtaccgcaatatgactacagtattgacaagagaatacctcgatgtacgccctgatgggtggttggactacgcagctatgagaattgcacagttgggagctgataagtgttataatcgtacattgtgtgaagagcatcttaatgtgattcttccagcgaagcctccttttcacccacggcagtttcataaatgcgctgttgttggaaattctggagatctgttaaaaacagagttcggagaagagattgacagtcatgatgctgtatttcgagacaatgaagctcctgtcaatgagaagtatgccaagtacgtaggagtgaaaagggatttccgactagttgttcgtggtgcagctcgtaacatgattaagattctcaatgggtctgacaatgaagtgcttataataaaaagtgtgacccaccgagacttcaatgaaatgataaagcgtataccaaaccctgtttatcttttccaaggaattgtcctacgcagaggtgccaaaggaaccgggatgaaatccattgaactagcattgtccatgtgcgatattgttgacatatatggtttcactgttgatcctggatacacagagtggacgcgttacttttccacgccaaggaaagggcacaacccacttcaaggaagggcatactaccaacttctagagtgtcttggtgtaatacggatccattctcccatgagatccgagagaaaggaggactggtcaagtgtgccaagtcgtgaaatgataagcagagctcacacagctgccttacgcctacaaaggagccaacagccaactagttcgaagagggatgggtcaggacaattcggtaattgcaaggtgtggggcgatgcagaccctacaaaaggacctgtctcgggatctccggacatgagtgagaccaggaagaagtcaaattacaagaaatgggaagtgatgccgtttaggagcttaagaaaggaagctagggatcactatatccaaatgaaaggcgtgtcacagtataagatggatggaaataagctggacgatctggtttgcgtgaggcatccactgaaattagatacgtaaatttttttatcatcttgtaaaggttcaatttcaataacatcttgagcttacaaagtaaaggttcctacatttcac

SEQ ID NO: 5 - DNA, 348 bp, Artificial Sequence
  Description of Artificial Sequence; note = synthetic construct
atgtgtgctagaatcgtgaagtattactcggtgaagaagttcttggaagagaagaaagcgaaagggtttgtcgattggagtaaagatcatgaaggctcgttgtttcactattcgtcgggtatgcaggctgtgatgcttgcagtggggatttgtgagaaagttagtgtcttcgggtttgggaagttgaattcaaccaagcaccattaccatactaatcagaaagcagagctgaagcttcatgactatgaagcagagtatagattgtatcgagacctggaaaacagtcccagggccattccattcttaccaaaagaattcaagatccctttggtacaagtttaccattga

SEQ ID NO: 6 - protein, 350 aa, Artificial Sequence
  Description of Artificial Sequence; note = synthetic construct
MAATPWYFVAVLLTILTSSQGILTTLSQSDGGYKYDYATVPFLAEFTVFKLIISGLFLWREMRTSSSTTSRITTDWKSVRLFVIPSLIYLIHNNVQFATLTYVDTSFTTYQIMGNLKIVTTGILFRLFLKRKLSKLQWMAIGLLAVGTTTSQVKGCGEASCDSLFTAFTPIQGYLLGILSAGLSALAGIYTEFLMKRNNDTLYWQNLQLYTFGSLFNVARLIADDFRHFTGFEKGPWWQRIFDGYSITTWLVVLNLGSTGLLVSWLMKYADNIVKVYSTSMAMLLTMVAFTSIYLFSFKPTLQLFLGIVICIMSLHMYFAPPHTLVDLPVTNEAHAKTLKQVVVEEKTDS

SEQ ID NO: 7 - protein, 419 aa, Artificial Sequence
  Description of Artificial Sequence; note = synthetic construct
MKNGIAECPACHSKLVSPGSKTISRAYDDHKIRVSSKQRVLNVLLFTVVGDCMLVGLQPVLVYMSKVDGKFNFSPISVNFLTEIAKVIFAIVMLLIQARHQKVGEKFTPLLSVSTFVQAARNNVLLAVPALLYAINNYLKFTMQLYFNPATVKMLSNLKVLVIAVLLFTKMVMKRRFSIIQWEALALLLIGISVNQLRSLPEGATAIGIPLATGAYVCTVIFVTVPSMFTASVFNEYALKSQYDTSIYLQNLFLYGYGAIFNFLGILGTVIYKGPGSFDILQGHSRATMFTFLILNNAAQGILSSFFFKYADTILKKYSSTVATIFTGIASAALFGHVITMNFLLGISIVFTFISMHQFFSPLAKARDEQQQNGNLELGNTKDTHRANESFINMAAGANEEASHRGESDDRFTTPLLPR

SEQ ID NO: 8 - protein, 454 aa, Artificial Sequence
  Description of Artificial Sequence; note = synthetic construct
MKLLHLIFLLALTTGISAVLIYIIGVSNLYESNRFTNEDLEALQSFTLQNGFQKCVSANGLGLQAAMGRDYCKVSINFPKDTVPKWKDPKSGELEGLSYEFDLCEAFTVATWEQVRNSSTILTKEYIDALPNGWEDYAWRRINKGIQLNRCQNKSLCIEKLSLVLPEFTTPPYFPRQFGRCAVIGNSGDLLKTKFGKEIDTYDTVLRENGAPIQNYKEYVGEKSTFRLFTLNRGSAKALDKVVELDEKKQEVLLVKTTIHDIMNKMIREVPIKNPVYLMLGASFGSAAKFTGTGLKALEFALSTCDSVDMYGFTVDPGYKEWTRYFSESRQGHTPLHGRAYYQMMECLGLFTIKIHSPMRADPNRVVKWVPSRSTIRSARIAAEKLLRRVGAGSADPLASCSIVKKRNKNKFTRPMVSHLRKPVSDHQKFVRSTSMYPVEHSPGHGQLCITPAD

SEQ ID NO: 9 - protein, 490 aa, Artificial Sequence
  Description of Artificial Sequence; note = synthetic construct
MRSHQAGRKLPLLQLLGCVAVFSVFVFTIQSSFFADNNRKLDLQPFTEDIQILSDFQSSVQQCVANRGLGLSAHIIDHCNLILKFPEGTNSTWYNAQFKVFEALEFFTKYNVCEAVLLWEQYRNMTTVLTREYLDVRPDGWLDYAAMRIAQLGADKCYNRTLCEEHLFTNVILPAKPPFHPRQFHKCAVVGNSGDLLKTEFGEEIDSHDAVFRDNEAPVNEKYAKYVGFTVKRDFRLVVRGAARNMIKILNGSDNEVLIIKSVTHRDFNEMIKRIPNPVYLFQGIVLRRFTGAKGTGMKSIELALSMCDIVDIYGFTVDPGYTEWTRYFSTPRKGHNPLQGRAYYQLLECFTLGVIRIHSPMRSERKEDWSSVPSREMISRAHTAALRLQRSQQPTSSKRDGSGQFGNCKVFTWGDADPTKGPVSGSPDMSETRKKSNYKKWEVMPFRSLRKEARDHYIQMKGVSQYKMDGNFTKLDDLVCVRHPLKLDT

SEQ ID NO: 10 - protein, 119 aa, Artificial Sequence
  Description of Artificial Sequence; note = synthetic construct
MCARIVKYYSVKKFLEEKKAKGFVDWSKDHEGSLFHYSSGMQAVMFTLAVGICEKVSVFGFGKLNSTKHHYHTNQKAELKLHDYEAEYRLYRDLENSPRAIPFLPKFTEFKIPLVQVYH

SEQ ID NO: 11 - DNA, 1362 bp, Artificial Sequence
  Description of Artificial Sequence; note = synthetic construct
ttggtgagaagagagactgaagaagagctacaaaaaatgagagagctcctgtgaagtgacgaagagacgaagaagaaagagaacattaatggcggctactccgtggtactttgtcgcagtacttctcaccatccttaccagctctcaggggattctaacaactctttctcaaagtgatggaggttataagtatgattatgctactgttccctttcttgctgaagtttttaagctgatcatttctggtttatttctctggagagagatgcggacatcttcctcaacaacttcaaggattactacggattggaagagtgtgcgattatttgttattccgtctcttatttatctgatacataacaatgtgcagtttgctacattgacttatgtggatacgtccacttatcagataatgggtaatttgaagattgtgaccactggcatactgtttaggttatttttgaaaaggaaactatctaaacttcagtggatggcaattggtcttttagctgttggaacaactaccagccaggttaagggttgtggagaagcttcttgtgattctttattcacggcaccaatacaaggttatttgctgggcatcctctcagctggtttgtccgcgctagctggaatctacacagagtttctgatgaagagaaacaatgacaccttatactggcaaaacttgcagttatatacgtttggttcacttttcaacgtagctcgacttatagcagatgatttcagacacgggtttgaaaagggtccttggtggcaacgtatctttgatgggtacagcatcaccacttggctggtagttctgaatctcggatccactggcttactggtctcgtggttaatgaagtatgcagacaacatcgtcaaggtttattctacatcaatggcgatgctacttactatggttgcatccatatacctttttagcttcaaaccgacactgcagcttttcttgggtattgtcatatgcatcatgtcactccatatgtactttgctcctccacacacgctcgtggacttgcccgtgacaaacgaagcacatgccaagaccttaaagcaagttgtcgtagaagaaaagacagattcttgaattaaattggacgaaaaggttagttgaacacatagaaaacaatttgctaagtaaatgaacacaatgatcttcttctagcttcatttctattagagaatgagaaacttggatgtgagttcttttgaagaaatgatctgatagtttgtttattcacgtaccgattcttttctttgtaaatgtacatcgaggtgcctagaaatagtaaattcgattgttgaaaaaaaaaaaaaaaaaaaaaaaaaaaaaaaaaa

SEQ ID NO: 12 - protein, 350 aa, Artificial Sequence
  Description of Artificial Sequence; note = synthetic construct
MAATPWYFVAVLLTVLTSSQGILTTLSQSDGGYKYDYATVPFLAEFTVFKLIISGLFLWREMRTSSSTTSRITTDWKSVRLFVIPSLIYLIHNNVQFATLTYVDTSFTTYQIMGNLKIVTTGILFRLFLKRKLSKLQWMAIGLLAVGTTTSQVKGCGEASCDSLFTAFTPIQGYLLGILSAGLSALAGIYTEFLMKRNNDTLYWQNLQLYTFGSLFNVARLIADDFRHFTGFEKGPWWQRIFDGYSITTWLVVLNLGSTGLLVSWLMKYADNIVKVYSTSMAMLLTMVAFTSIYLFSFKPTLQLFLGIVICIMSLHMYFAPPHTLVDLPVTNEAHAKTLKQVVVEEKTDS

SEQ ID NO: 13 - DNA, 1362 bp, Artificial Sequence
  Description of Artificial Sequence; note = synthetic construct
ttggtgagaagagagactgaagaagagctacaaaaaatgagagagctcctgtgaagtgacgaagagacgaagaagaaagagaacattaatggcggctactccgtggtactttgtcgcagtacttctcaccgttcttaccagctctcaggggattctaacaactctttctcaaagtgatggaggttataagtatgattatgctactgttccctttcttgctgaagtttttaagctgatcatttctggtttatttctctggagagagatgcggacatcttcctcaacaacttcaaggattactacggattggaagagtgtgcgattatttgttattccgtctcttatttatctgatacataacaatgtgcagtttgctacattgacttatgtggatacgtccacttatcagataatgggtaatttgaagattgtgaccactggcatactgtttaggttatttttgaaaaggaaactatctaaacttcagtggatggcaattggtcttttagctgttggaacaactaccagccaggttaagggttgtggagaagcttcttgtgattctttattcacggcaccaatacaaggttatttgctgggcatcctctcagctggtttgtccgcgctagctggaatctacacagagtttctgatgaagagaaacaatgacaccttatactggcaaaacttgcagttatatacgtttggttcacttttcaacgtagctcgacttatagcagatgatttcagacacgggtttgaaaagggtccttggtggcaacgtatctttgatgggtacagcatcaccacttggctggtagttctgaatctcggatccactggcttactggtctcgtggttaatgaagtatgcagacaacatcgtcaaggtttattctacatcaatggcgatgctacttactatggttgcatccatatacctttttagcttcaaaccgacactgcagcttttcttgggtattgtcatatgcatcatgtcactccatatgtactttgctcctccacacacgctcgtggacttgcccgtgacaaacgaagcacatgccaagaccttaaagcaagttgtcgtagaagaaaagacagattcttgaattaaattggacgaaaaggttagttgaacacatagaaaacaatttgctaagtaaatgaacacaatgatcttcttctagcttcatttctattagagaatgagaaacttggatgtgagttcttttgaagaaatgatctgatagtttgtttattcacgtaccgattcttttctttgtaaatgtacatcgaggtgcctagaaatagtaaattcgattgttgaaaaaaaaaaaaaaaaaaaaaaaaaaaaaaaaaa

SEQ ID NO: 14 - DNA, 1362 bp, Artificial Sequence
  Description of Artificial Sequence; note = synthetic construct
ttggtgagaagagagactgaagaagagctacaaaaaatgagagagctcctgtgaagtgacgaagagacgaagaagaaagagaacattaatggcggctactccgtggtactttgtcgcagtacttctcaccgtccttaccagctctcaggggattctaacaactctttctcaaagtgatggaggttataagtatgattatgctactgttccctttcttgctgaagtttttaagctgatcatttctggtttatttctctggagagagatgcggacatcttcctcaacaacttcaaggattactacggattggaagagtgtgcgattatttgttattccgtctcttatttatctgatacataacaatgtgcagtttgctacattgacttatgtggatacgtccacttatcagataatgggtaatttgaagattgtgaccactggcatactgtttaggttatttttgaaaaggaaactatctaaacttcagtggatggcaattggtcttttagctgttggaacaactaccagccaggttaagggttgtggagaagcttcttgtgattctttattcacggcaccaatacaaggttatttgctgggcatcctctcagctggtttgtccgcgctagctggaatctacacagagtttctgatgaagagaaacaatgacaccttatactggcaaaacttgcagttatatacgtttggttcacttttcaacgtagctcgacttatagcagatgatttcagacacgggtttgaaaagggtccttggtggcaacgtatctttgatgggtacagcatcaccacttggctggtagttctgaatctcggatccactggcttactggtctcgtggttaatgaagtatgcagacaacatcgtcaaggtttattctacatcaatggcgatgctacttactatggttgcatccatatacctttttagcttcaaaccgacactgcagcttttcttgggtattgtcatatgcatcatgtcactccatatgtactttgctcctccacacacgctcgtggacttgcccgtgacaaacgaagcacatgccaagaccttaaagcaagttgtcgtagaagaaaagacagattcttgaattaaattggacgaaaaggttagttgaacacatagaaaacaatttgctaagtaaatgaacacaatgatcttcttctagcttcatttctattagagaatgagaaacttggatgtgagttcttttgaagaaatgatctgatagtttgtttattcacgtaccgattcttttctttgtaaatgtacatcgaggtgcctagaaatagtaaattcgattgttgaaaaaaaaaaaaaaaaaaaaaaaaaaaaaaaaaa

SEQ ID NO: 15 - DNA, 1472 bp, Homo sapiens
gtggagggtcgcggcaggaacaagtgcgggggcggggcctcaaagccccgcccccaggcccagcggaaccctgaagaggagggggagggtgagccgggcgcggctgtgtcagctgagtgacagtcacgggaccgcgaccaactctgtctggcttcctagaggagcaggagggcggggcaagggtctcccagcgcgacaggacatggagaaagagcgagagactctgcaggcctggaaggagcgcgtggggcaggagctggaccgcgtggtggctttctggatggagcactcccacgaccaggagcacgggggcttcttcacgtgccttggccgcgaggggcgggtgtatgatgacctcaagtatgtgtggctgcaggggaggcaggtatggatgtattgtcgcctgtaccgcactttcgagcgcttccgccatgctcagcttctggacgcagcaaaagcaggtggtgagttcttgctgcggtatgcccgggtggcacctcctggcaagaagtgtgcctttgtgctgactcgggacggccgcccggtcaaggtgcagcgaaccatcttcagtgagtgtttctacaccatggccatgaacgagctgtggagagccacaggggaagtgcggtaccagacggaagcggtggagatgatggatcagatcgtccactgggtgcaggaggacgcgtcgggactgggccggccccagctccagggggccccggctgcggagcccatggcggtgcccatgatgctactgaacctggtggagcagctcggggaggcagatgaggagctggcgggcaaatacgcagagctgggggactggtgcgcccggaggattctgcagcacgtgcagagggatggacaagctgtgctggagaatgtgtcagagggtggcaaggaacttcctggctgcctggggagacagcagaacccaggccacacgctggaagccggctggtttctgctccgtcattgcattcggaaaggcgaccccgaacttcgagcccacgtgattgacaagttcctattgttgcccttccactccggatgggaccctgaccacggaggcctcttttacttccaggatgctgataacttctgccccacccagctggagtgggccatgaagctctggtggccacacagtgaagccatgattgccttcctcatgggttacagtgacagtggggaccctgtgctgctgcgcctcttctaccaagtggctgagtacaccttccgccagtttcgcgatcccgagtacggggaatggtttggctacctgagccgagagggcaaggtggccctctccatcaagggaggtcctttcaaaggctgcttccacgtgccgcggtgcctagccatgtgcgaggagatgctgggcgccctgctgagccgccccgcccccgccccctcccccgcccccacccccgcctgccgaggcgcggaataaaggctgagtccgctcc

SEQ ID NO: 16 - protein, 417 aa, Homo sapiens
MEKERETLQAWKERVGQELDRVVAFWMEHSHDQEHGGFFTCLGREGRVYDDLKYVWLQGRQVWMYCRLYRTFERFRHAQLLDAAKAGGEFLLRYARVAPPGKKCAFVLTRDGRPVKVQRTIFSECFYTMAMNELWRATGEVRYQTEAVEMMDQIVHWVQEDASGLGRPQLQGAPAAEPMAVPMMLLNLVEQLGEADEELAGKYAELGDWCARRILQHVQRDGQAVLENVSEGGKELPGCLGRQQNPGHTLEAGWFLLRHCIRKGDPELRAHVIDKFLLLPFHSGWDPDHGGLFYFQDADNFCPTQLEWAMKLWWPHSEAMIAFLMGYSDSGDPVLLRLFYQVAEYTFRQFRDPEYGEWFGYLSREGKVALSIKGGPFKGCFHVPRCLAMCEEMLGALLSRPAPAPSPAPTPACRGAE

SEQ ID NO: 17 - DNA, 3760 bp, Homo sapiens
gctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttcgctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtcccagctactcgggaggctgaggcaggagaatggcatgaaccccaggctggagtgcagtggctcaatgcaacctctgccccccaagttcaagtgattctcctgcctcagcctcttgagtagctgggattacaggcacatgcaccacgcccggctgatttttttttgtatttttagtagacagggtttcaccatgttggccaggctggtcttgaactcctgacctcaggtgatccgcccgcctctgcctcccaaagtgctgggattacaggtgtaagccatcacacctggccctagtgacaggtttttatgggtacttttagatgatctaagaaatcatgtgcatatatctttcagatttctattttgggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccagggcctggttattgttcagacattccatggtgattcctgggaaggaagcttggctgctcagtttctgagtctggggtgagataatgttctgggaagggacatctgttctttggtgtaatctctcatggtgaaatctgctctgtacatcagacaattgcattgctaccaagtttcataccaaatatttgaaaggatggtattgaatctaaaaccaaatattagtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaaaaaaaaaa

SEQ ID NO: 18 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 19 - DNA, 1170 bp, Homo sapiens
agagtagaggcggcggcggcggcggccggacccagactggtagtgaggctttggaccccgagccgctgcaatgccgctggagctggagctgtgtcccgggcgctgggtgggcgggcaacacccgtgcttcatcattgccgagatcggccagaaccaccagggcgacctggacgtagccaagcgcatgatccgcatggccaaggagtgtggggctgattgtgctaagttccagaagagtgagctagaattcaagtttaatcggaaagccttggagaggccatacacctcgaagcattcctgggggaagacgtacggggagcacaaacgacatctggagttcagccatgaccagtacagggagctgcagaggtacgccgaggaggttgggatcttcttcactgcctctggcatggatgagatggcagttgaattcctgcatgaactgaatgttccatttttcaaagttggatctggagacactaataattttccttatctggaaaagacagccaaaaaaggtcgcccaatggtgatctccagtgggatgcagtcaatggacaccatgaagcaagtttatcagatcgtgaagcccctcaaccccaacttctgcttcttgcagtgtaccagcgcatacccgctccagcctgaggacgtcaacctgcgggtcatctcggaatatcagaagctctttcctgacattcccatagggtattctgggcatgaaacaggcatagcgatatctgtggccgcagtggctctgggggccaaggtgttggaacgtcacataactttggacaagacctggaaggggagtgaccactcggcctcgctggagcctggagaactggccgagctggtgcggtcagtgcgtcttgtggagcgtgccctgggctccccaaccaagcagctgctgccctgtgagatggcctgcaatgagaagctgggcaagtctgtggtggccaaagtgaaaattccggaaggcaccattctaacaatggacatgctcaccgtgaaggtgggtgagcccaaaggctatcctcctgaagacatctttaatctagtgggcaagaaggtcctggtcactgttgaagaggatgacaccatcatggaagaattggtagataatcatggcaaaaaaatcaagtcttaaaaataaagtgccattctctg

SEQ ID NO: 20 - protein, 359 aa, Homo sapiens
MPLELELCPGRWVGGQHPCFIIAEIGQNHQGDLDVAKRMIRMAKECGADCAKFQKSELEFKFNRKALERPYTSKHSWGKTYGEHKRHLEFSHDQYRELQRYAEEVGIFFTASGMDEMAVEFLHELNVPFFKVGSGDTNNFPYLEKTAKKGRPMVISSGMQSMDTMKQVYQIVKPLNPNFCFLQCTSAYPLQPEDVNLRVISEYQKLFPDIPIGYSGHETGIAISVAAVALGAKVLERHITLDKTWKGSDHSASLEPGELAELVRSVRLVERALGSPTKQLLPCEMACNEKLGKSVVAKVKIPEGTILTMDMLTVKVGEPKGYPPEDIFNLVGKKVLVTVEEDDTIMEELVDNHGKKIKS

SEQ ID NO: 21 - DNA, 1741 bp, Homo sapiens
gatcgggcggcgccgagctgaggtggtgagggactagctcccggatgtggagaagctggggagaaggcgtgggaggaagatggactcggtggagaagggggccgccacctccgtctccaacccgcgggggcgaccgtcccggggccggccgccgaagctgcagcgcaactctcgcggcggccagggccgaggtgtggagaagcccccgcacctggcagccctaattctggcccggggaggcagcaaaggcatccccctgaagaacattaagcacctggcgggggtcccgctcattggctgggtcctgcgtgcggccctggattcaggggccttccagagtgtatgggtttcgacagaccatgatgaaattgagaatgtggccaaacaatttggtgcacaagttcatcgaagaagttctgaagtttcaaaagacagctctacctcactagatgccatcatagaatttcttaattatcataatgaggttgacattgtaggaaatattcaagctacttctccatgtttacatcctactgatcttcaaaaagttgcagaaatgattcgagaagaaggatatgattctgttttctctgttgtgagacgccatcagtttcgatggagtgaaattcagaaaggagttcgtgaagtgaccgaacctctgaatttaaatccagctaaacggcctcgtcgacaagactgggatggagaattatatgaaaatggctcattttattttgctaaaagacatttgatagagatgggttacttgcagggtggaaaaatggcatactacgaaatgcgagctgaacatagtgtggatatagatgtggatattgattggcctattgcagagcaaagagtattaagatatggctattttggcaaagagaagcttaaggaaataaaacttttggtttgcaatattgatggatgtctcaccaatggccacatttatgtatcaggagaccaaaaagaaataatatcttatgatgtaaaagatgctattgggataagtttattaaagaaaagtggtattgaggtgaggctaatctcagaaagggcctgttcaaagcagacgctgtcttctttaaaactggattgcaaaatggaagtcagtgtatcagacaagctagcagttgtagatgaatggagaaaagaaatgggcctgtgctggaaagaagtggcatatcttggaaatgaagtgtctgatgaagagtgcttgaagagagtgggcctaagtggcgctcctgctgatgcctgttctactgcccagaaggctgttggatacatttgcaaatgtaatggtggccgtggtgccatccgagaatttgcagagcacatttgcctactaatggaaaaggttaataattcatgccaaaaatagaaattagcgtaatattgagaaaaaaatgatacagccttcttcagccagtttgcttttatttttgattaagtaaattccatgttgtaatgttacagagagtgtgatttggtttgtgatatatatatattgtgctctacttttctctttacgcaagataattatttagagactgattacagtctttctcagatttttagtaaatgcaagtaagaacatcatcaaagttcactttgtattgtaccctgtaaaactgtgtgtttgtgtgctttcaaagatgttgggattttatttatctggggacagtgtgtatggtaagacatgcccttctattaataaaactacatttctcaaacttga

SEQ ID NO: 22 - protein, 434 aa, Homo sapiens
MDSVEKGAATSVSNPRGRPSRGRPPKLQRNSRGGQGRGVEKPPHLAALILARGGSKGIPLKNIKHLAGVPLIGWVLRAALDSGAFQSVWVSTDHDEIENVAKQFGAQVHRRSSEVSKDSSTSLDAIIEFLNYHNEVDIVGNIQATSPCLHPTDLQKVAEMIREEGYDSVFSVVRRHQFRWSEIQKGVREVTEPLNLNPAKRPRRQDWDGELYENGSFYFAKRHLIEMGYLQGGKMAYYEMRAEHSVDIDVDIDWPIAEQRVLRYGYFGKEKLKEIKLLVCNIDGCLTNGHIYVSGDQKEIISYDVKDAIGISLLKKSGIEVRLISERACSKQTLSSLKLDCKMEVSVSDKLAVVDEWRKEMGLCWKEVAYLGNEVSDEECLKRVGLSGAPADACSTAQKAVGYICKCNGGRGAIREFAEHICLLMEKVNNSCQK

SEQ ID NO: 23 - protein, 346 aa, Artificial Sequence
  Description of Artificial Sequence; note = synthetic construct
MKEIKIQNIIISEEKAPLVVPEIGINHNGSLELAKIMVNAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVALGACVLERHFTDSMHRSGPDIVCSMDTQALKELIIQSEQMAIMRGNNESKKAAKQEQVTIDFAFASVVSIKDIKKGEVLSMDNIWVKRPGLGGISASEFENILGKKALRDIENDTQLSYEDFA

SEQ ID NO: 24 - protein, 439 aa, Artificial Sequence
  Description of Artificial Sequence; note = synthetic construct
MRVLPLALAAAIFSGVTAILVYLSGLSSYGGARVSDADLAALGALQSGFSKCVDANGLGLKAIPGEDYCRVVIQYPSDTDSKWKDPKTGEPEGLSFEFNLCEAVASWEQVRNSTTILTKEYIDALPNGWEEYAWRRINKGIHLNKCQNRTLCMEKLSLVLPETPPYVPRQFGRCAVVGNSGDLLKTKFGDEIDSYDVVIRENGAPIQNYTEYVGTKSTFRLLNRGSAKALDKVVELDETKKEALIVKTTIHDIMNQMIREIPITNPVYLMLGTSFGSSAKGTGLKALEFALSMCDSVDMYGFTVDPGYKEWTRYFSESRKGHTPLHGRAYYQMMECLGLVKIHSPMRGDPGRVVKWAPTKDTIEAARVASEKLLKRPGAGSEGPLSSCTMIKKREKGKTPKRSVVRHAALKHLEYMRGATRYPLERNAGGGYLCMINER

SEQ ID NO: 25 - protein, 438 aa, Artificial Sequence
  Description of Artificial Sequence; note = synthetic construct
MRVLPLALAAAIFSGVTAILVYLSGLSSYGARVSDADLAALGALQSGFSKCVDANGLGLKAIPGEDYCRVVIQYPSDTDSKWKDPKTGEPEGLSFEFNLCEAVASWEQVRNSTTILTKEYIDALPNGWEEYAWRRINKGIHLNKCQNRTLCMEKLSLVLPETPPYVPRQFGRCAVVGNSGDLLKTKFGDEIDSYDVVIRENGAPIQNYTEYVGTKSTFRLLNRGSAKALDKVVELDETKKEALIVKTTIHDIMNQMIREIPITNPVYLMLGTSFGSSAKGTGLKALEFALSMCDSVDMYGFTVDPGYKEWTRYFSESRKGHTPLHGRAYYQMMECLGLVKIHSPMRGDPGRVVKWAPTKDTIEAARVASEKLLKRPGAGSEGPLSSCTMIKKREKGKTPKRSVVRHAALKHLEYMRGATRYPLERNAGGGYLCMINER

SEQ ID NO: 26 - protein, 439 aa, Artificial Sequence; identical to SEQ ID NO: 24
  Description of Artificial Sequence; note = synthetic construct

SEQ ID NO: 27 - protein, 393 aa, Artificial Sequence
  Description of Artificial Sequence; note = synthetic construct
MKRPLRRPFAVLLFVVLCAAASFPSVLRRSVGPAPVLAPLPPLDPARLNATLLRLAAADPSEAPLRRDVDDLLEGRLPASSARARAWRLRGDRLHLHLRHHQFPVYRRGHHPDHDHDPLLHPLPRQELHLDPSLRRALRSWHRLRRHDPGVLRNLPSLLSLPGRIPSCAVVGNSGILLGASHGALIDSHAAVFRLNNARISGFAANVGAKTNLSFINSNVLHLCARRPNCFCHPYGDGVPILLYICQAAHFLDVASCNASSRSLHAASISVTDPRLDVLCARIVKYYSLRRFVAETGRAAEEWSSTRDAAMFHYSSGMQAIMVAVGVCDRVSVFGFGKAADAKHHYHSNQKAELDLHDYKAEYAFYRDLADRPEVVPFLNDAGIAVPPVVFYH

SEQ ID NO: 28 - protein, 393 aa, Artificial Sequence; identical to SEQ ID NO: 27
  Description of Artificial Sequence; note = synthetic construct

SEQ ID NO: 29 - protein, 393 aa, Artificial Sequence; identical to SEQ ID NO: 27
  Description of Artificial Sequence; note = synthetic construct

SEQ ID NO: 30 - protein, 384 aa, Artificial Sequence
  Description of Artificial Sequence; note = synthetic construct
MKRRHWSHPSCGLLLLVAVFCLLLVFRCSQLRHSGDGAAAAAPDGGAGRNDGDDVDERLVELAAVDPAAMAVLQAAKRLLEGNLARAPERHRDVALRGLREWVGKQERFDPGVMSELVELIKRPIDRYNGDGGGGGEGEGRRYASCAVVGNSGILLAAEHGELIDGHELVVRLNNAPAGDGRYARHVGARTGLAFLNSNVLSQCAVPRRGACFCRAYGEGVPILTYMCNAAHFVEHAVCNNASSSSSGAADATAAAPVIVTDPRLDALCARIVKYYSLRRFARETGRPAEEWARRHEEGMFHYSSGMQAVVAAAGVCDRVSVFGFGKDASARHHYHTLQRRELDLHDYEAEYEFYRDLESRPEAIPFLRQRNSGFRLPPVSFYR

SEQ ID NO: 31 - protein, 384 aa, Artificial Sequence; identical to SEQ ID NO: 30
  Description of Artificial Sequence; note = synthetic construct

SEQ ID NO: 32 - protein, 384 aa, Artificial Sequence; identical to SEQ ID NO: 30
  Description of Artificial Sequence; note = synthetic construct